(19) United States
(12) Patent Application Publication
(10) Pub. No.: US 2012/0022197 A2
(43) Pub. Date: Jan. 26, 2012

(54) PROCESS FOR MAKING MIXED TRIGLYCERIDE PLASTICIZER FROM BENZOIC AND TOLUIC ACID

(76) Inventors: Jihad Dakka, Whitehouse Station, NJ (US); Edmund Mozeleski, Califon, NJ (US); Lisa Baugh, Ringoes, NJ (US); Allen Godwin, Seabrook, TX (US); Manika Varma-Nair, Warren, NJ (US)

(21) Appl. No.: 12/878,166

(22) Filed: Sep. 9, 2010

(51) Int. Cl. C08K 5/101; C09K 3/00; C07C 69/76; C08K 5/12; C08K 5/11

(52) U.S. Cl. 524/285; 524/287; 524/296; 524/314; 524/295; 560/76; 252/182.28

(57) ABSTRACT

Provided are compositions, processes for making, and processes for using mixed triglycerides as plasticizers. Triglyceride plasticizers can be produced by recovery of linear or branched C₄ to C₁₃ aldehydes from a hydroformylation product, oxidation to linear or branched C₄ to C₁₃ acids with oxygen and/or air, recovery of the resulting acids, combining the linear or branched C₄ to C₁₃ acid with benzoic acid, toluic acid or a combination thereof to form a mixed acid blend, and esterification of the mixed acid blend with glycerol, wherein the total carbon number of the triester groups ranges from 20 to 25 and includes from 1 to 2 aryl groups for greater than or equal to 45 wt % of the plasticizer. Such plasticizers can be phthalate-free and provide outstanding properties including a suitable melting or pour point, glass transition temperature, low volatility, increased compatibility, increased hydrolytic stability, and excellent low temperature properties in a range of polymeric resins.

### CROSS-REFERENCE TO RELATED APPLICATIONS

This is a Non-Provisional Application that claims priority to U.S. Provisional Application No. 61/279,671 filed on Oct. 23, 2009 and herein incorporated by reference in its entirety.

### FIELD

The present disclosure relates to a process for making mixed triglycerides based on linear or branched alkyl groups and aryl groups, useful as plasticizers and viscosity depressants for a wide range of polymer resins.

### BACKGROUND

Plasticizers are incorporated into a resin (usually a plastic or elastomer) to increase the flexibility, workability, or distensibility of the resin. The largest use of plasticizers is in the production of “plasticized” or flexible polyvinyl chloride (PVC) products. Typical uses of plasticized PVC include films, sheets, tubing, coated fabrics, wire and cable insulation and jacketing, toys, flooring materials such as vinyl sheet flooring or vinyl floor tiles, adhesives, sealants, inks, and medical products such as blood bags and tubing, and the like.

Other polymer systems that use small amounts of plasticizers include polyvinyl butyral, acrylic polymers, poly(vinylidene chloride), nylon, polyolefins, polyurethanes, and certain fluoroplastics. Plasticizers can also be used with rubber (although often these materials fall under the definition of extenders for rubber rather than plasticizers). A listing of the major plasticizers and their compatibilities with different polymer systems is provided in “Plasticizers,” A. D. Godwin, in Applied Polymer Science 21st Century, edited by C. D. Craver and C. E. Carraher, Elsevier (2000); pp. 157-175.

Plasticizers can be characterized on the basis of their chemical structure. The most important chemical class of plasticizers is phthalic acid esters, which accounted for about 85% worldwide of PVC plasticizer usage in 2002. However, in the recent past there as been an effort to decrease the use of phthalate esters as plasticizers in PVC, particularly in end uses where the product contacts food, such as bottle cap liners and sealants, medical and food films, or for medical examination gloves, blood bags, and IV delivery systems, flexible tubing, or for toys, and the like. For these and most other uses of plasticized polymer systems, however, a successful substitute for phthalate esters has heretofore not materialized.

One such suggested substitute for phthalates are esters based on cyclohexanoic acid. In the late 1990's and early 2000's, various compositions based on cyclohexanoate, cyclohexanedioates, and cyclohexanepolyoate esters were said to be useful for a range of goods from semi-rigid to highly flexible materials. See, for instance, WO 99/32427, WO 2004/046078, WO 2003/029339, WO 2004/046078, U.S. Application No. 2006-0247461, and U.S. Pat. No. 7,297,738.

Other suggested substitutes include esters based on benzoic acid (see, for instance, U.S. Pat. No. 6,740,254, and also co-pending, commonly-assigned, U.S. Patent Application Ser. No. 61/040,480, filed on Mar. 28, 2008 and polyketones, such as described in U.S. Pat. No. 6,777,514; and also co-pending, commonly-assigned, U.S. patent application Ser. No. 12/058,397 filed on Mar. 28, 2008. Epoxidized soybean oil, which has much longer alkyl groups (C₁₆ to C₁₈) has been tried as a plasticizer, but is generally used as a PVC stabilizer. Stabilizers are used in much lower concentrations than plasticizers.

Typically, the best that has been achieved with substitution of the phthalate ester with an alternative material is a flexible PVC article having either reduced performance or poorer processability. Thus, heretofore efforts to make phthalate-free plasticizer systems for PVC have not proven to be entirely satisfactory, and this is still an area of intense research.

Plasticizers based on triglycerides have been tried in the past, but they have mostly been based on natural triglycerides from various vegetable oils. The alkyl groups on these natural triglycerides are linear, and can cause compatibility problems when the alkyl chain is too long.

“Structural Expressions of Long-Chain Esters on Their Plasticizing Behavior in Poly(Vinyl Chloride)”, H. K. Shobha and K. Kishore, Macromolecules 1992, 25, 6765-6769, reported the influence of branching and molecular weight in long-chain esters in PVC. Triglycerides (TGE's) having linear alkyl groups were studied.

“A Method for Determining compatibility Parameters of Plasticizers for Use in PVC Through Use of Torsional Modulus”, G. R. Riser and W. E. Palm, Polymer Engineering and Science, April 1967, 110-114, also investigate the use of triglycerides and their plasticizing behavior with PVC, including tri-iso-valerin (3-methyl butanoate) triglyceride. It was reported that “these materials have volatilities that are much too high for good long-time permanence”.

Nagai et al. in U.S. Pat. No. 5,248,531, teaches the use of articles comprising vinyl chloride-type resins (among others) using triglyceride compounds as a hemolysis depressant, and also comprising 10 to 45 wt % of plasticizers selected from trialkyl trimellitates, di-normal alkyl phthalates, and tetraalkyl pyromellitates. The alkyl chains of the acid-derived moiety R¹-R³ in the structure below, formula (I), are independently an aliphatic hydrocarbon group of 1 to 20 carbon atoms and in embodiments at least one of the alkyl chains is branched. One specific triglyceride disclosed is glyceryl tri-2-ethylhexanoate.

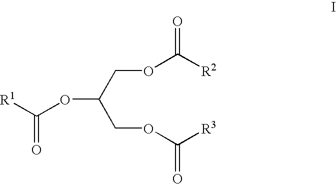

Zhou et al. discloses, in U.S. Pat. Nos. 6,652,774; 6,740,254; and 6,811,722; phthalate-free plasticizers comprising a mixture of different triesters of glycerin, preferably wherein the phthalate-free plasticizer is formed by a process of esterifying glycerin with a mixture comprising a mixture of alkyl acids and aryl acids. Zhou et al. also discloses that glyceryl tribenzoate and glyceryl tri(2-ethyl)hexanoate have not been used as primary plasticizers in vinyl polymers, such as PVC because they are known to be incompatible with such resins.

Nielsen et al., in U.S. Pat. No. 6,734,241, teach a composition comprising a thermoplastic polymer as in formula (I) above, wherein at least one of the R groups is an alkyl group having from 1-5 carbon atoms and at least one of the R groups is a saturated branched alkyl group having from 9 to 19 carbon atoms and a hydrophilic group.

Among the problems presented by the aforementioned triglycerides is they cannot be made conveniently and thus generally are quite expensive and/or are specialty chemicals not suitable as replacements for phthalates from an economic standpoint and/or are not as compatible with the range of polymer systems that phthalates are compatible with, and thus are not viable replacements for phthalates from a physical property standpoint.

For instance, some synthesis methods involve at least two separate steps, such as where the glycerol is first partially esterified with the C₁₀ to C₂₀ branched chain acyl halide, and then reacted with acetic acid or acetic anhydride to provide the remaining groups.

Other syntheses involving mixed acid feeds will require addition of a hydrocarbon solvent for azeotropic distillation of the water to drive the esterification reaction to completion (as measured by the hydroxyl number of the ester, which is a measure of the amount of unreacted OH groups), due to the spread in boiling points between the mixed acids. In addition, the use of mixed acid feedstock such as cited in Zhou et al. and in Nielsen et al. can reduce the capability of recycling unreacted acids.

Triglycerides based on acids derived from natural products will be limited to naturally occurring linear alkyl groups with even carbon numbers, which offer very little flexibility in designing an appropriate plasticizer system for a given polymer system.

Thus what is needed is a method of making a general purpose non-phthalate plasticizer providing a plasticizer having suitable melting or pour point, glass transition temperature, increased compatibility, good performance and low temperature properties.

Triglycerides produced by esterification of glycerol with a combination of acids derived from the hydroformylation and subsequent oxidation of C₃ to C₁₂ olefins provide for triglycerides having excellent compatibility with a wide variety of resins. Esterification of glycerol using a combination of these acids eliminates many of the aforementioned problems, and enables high yields of the glycerol triesters to be obtained, which have excellent compatibility with vinyl polymers. These acids are generally alkyl acids that are linear, branched or a combination thereof. However, it is generally recognized in the art that plasticizers produced from linear or branched alkyl acids have poor chemical stability toward hydrolysis.

Hence, there is a need for a process to produce triglycerides having improved hydrolytic stability, and for plasticized polymer compositions containing these more hydrolytically stable triglycerides.

### SUMMARY

The present disclosure is directed to a process for producing a plasticizer including: (i) recovering at least one linear C₄ to C₁₃ aldehyde, one branched C₄ to C₁₃ aldehyde, or a combination thereof from a hydroformylation product; (ii) oxidizing the linear, branched or combination thereof C₄ to C₁₃ aldehyde to form a linear, branched or combination thereof C₄ to C₁₃ acid; (iii) combining the linear, branched or combination thereof C₄ to C₁₃ acid with benzoic acid, toluic acid or a combination thereof at a molar ratio ranging from 0.25:1 to 4:1 to form a mixed acid blend; (iv) esterifying the mixed acid blend with a glycerol to yield a linear alkyl-aryl triglyceride, a branched alkyl-aryl triglyceride, or a combination thereof; and (v) purifying the linear, branched or combination thereof alkyl-aryl triglyceride to form a plasticizer, wherein the total carbon number of the triester groups ranges from 20 to 25 and includes from 1 to 2 aryl groups for greater than or equal to 45 wt % of the plasticizer.

The present disclosure is also directed to a process for producing a plasticizer including: (i) recovering an aldehyde/alcohol mixture including at least one linear C₄ to C₁₃ aldehyde, one branched C₄ to C₁₃ aldehyde, or a combination thereof and at least one linear C₄ to C₁₃ alcohol, one branched C₄ to C₁₃ alcohol, or a combination thereof from a hydroformylation process; (ii) oxidizing the aldehyde/alcohol mixture to form a linear, branched or combination thereof C₄ to C₁₃ acid; (iii) combining the linear, branched or combination thereof C₄ to C₁₃ acid with benzoic acid, toluic acid or a combination thereof at a molar ratio ranging from 0.25:1 to 4:1 to form a mixed acid blend; (iv) esterifying the mixed acid blend with glycerol to yield a linear alkyl-aryl triglyceride, a branched alkyl-aryl triglyceride, or a combination thereof; and (v) purifying the linear, branched or combination thereof alkyl-aryl triglyceride to form a plasticizer, wherein the total carbon number of the triester groups ranges from 20 to 25 and includes from 1 to 2 aryl groups for greater than or equal to 45 wt % of the plasticizer.

The present disclosure is also directed to a plasticizer comprising a triglyceride according to the formula: 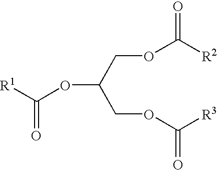
wherein each of R¹, R², and R³ are independently selected from a combination of C₃ to C₁₂ linear or branched alkyl groups and aryl groups, and wherein the total carbon number of the triester groups ranges from 20 to 25, and wherein the aryl groups are selected from benzoate groups, toluate groups and combinations thereof, and wherein the molar ratio of C₃ to C₁₂ linear or branched alkyl groups to benzoate and/or toluate groups in the triglyceride ranges from 0.5:1 to 2:1.

The present disclosure is still further directed to resin compositions, plastisols and articles comprising the above plasticizer compositions to provide phthalate-free plasticizers, resin compositions, plastisols and articles.

These and other objects, features, and advantages will become apparent as reference is made to the following detailed description, embodiments, examples, and appended claims.

### BRIEF DESCRIPTION OF THE DRAWINGS

In the accompanying drawings, like reference numerals are used to denote like parts throughout the several views.

FIG. 1 is a schematic representation of a process according to a preferred embodiment of the invention.

FIG. 2 shows the product compositions in weight % of the products prepared in Examples 2, 3, 5, and 7 (TGn=trialkyl triglyceride, TGben=tribenzyl triglyceride, TG2n/ben=alkyl/alkyl/benzoic triglyceride, TG2ben/n=benzoic/benzoic/alkyl triglyceride).

FIG. 3 shows hydrolytic stability test results for the mixed Oxo C₇-benzoic triglyceride (“TG7-ben”) prepared in Example 5; for comparison, results for DINP and the analogous tri-C₇ triglyceride (“TG7”) are also shown as discussed in Example 11.

FIG. 4 is an overlay plot of the DMTA tan delta curve and the DSC curve for PVC plasticized with the triglyceride ester prepared in Example 1, showing correlation between the DSC and DMTA glass transition onsets.

FIG. 5 is an overlay plot of the Dynamic Mechanical Thermal Analysis (DMTA) storage modulus curves for (a) neat PVC, (b) PVC plasticized with the commercial phthalate DINP, and (c) PVC plasticized with the triglyceride ester prepared in Example 1 (“Oxo C₉ Benz TG”).

FIG. 6 is an overlay plot of DMTA tan delta curves for (a) neat PVC, (b) PVC plasticized with the commercial phthalate DINP, and (c) PVC plasticized with the triglyceride ester prepared in Example 1 (“Oxo C₉ Benz TG”).

### DETAILED DESCRIPTION

The present disclosure provides triglycerides and methods of making triglycerides for use as plasticizers for polymer resins via esterification of a mixed acid blend with glycerol (a polyol) to yield a linear alkyl-aryl triglyceride, a branched alkyl-aryl triglyceride, or a combination thereof wherein the mixed acid blend includes a mixture of a linear, branched or combination thereof C₄ to C₁₃ acid with benzoic acid or toluic acid or a mixture of the two thereof.

U.S. Provisional Application No. 61/203,626 filed on Dec. 24, 2008, herein incorporated by reference in its entirety, discloses mixed triglyceride compositions, processes for making, and processes for using triglycerides as plasticizers. In one form of the process for making such mixed triglycerides, the steps include: (i) recovering at least one linear C₄ to C₁₃ aldehyde, one branched C₄ to C₁₃ aldehyde, or a combination thereof from a hydroformylation product; (ii) oxidizing the linear, branched or combination thereof C₄ to C₁₃ aldehyde to form a linear, branched or combination thereof C₄ to C₁₃ acid; (iii) esterifying the linear, branched or combination thereof C₄ to C₁₃ acid with a polyol to yield a linear alkyl triglyceride, a branched alkyl triglyceride, or a combination thereof; and (iv) purifying the linear, branched or combination thereof alkyl triglyceride to form a plasticizer, wherein the total carbon number of the triester groups ranges from 20 to 25 and includes from 1 to 2 aryl groups for greater than or equal to 45 wt % of the plasticizer. Pure glycerol is one of polyols that may be used in esterfying the linear, branched or combination thereof C₄ to C₁₃ acid to yield a linear alkyl triglyceride, a branched alkyl triglyceride, or a combination thereof.

U.S. Provisional Application No. 61/211,279, filed on Mar. 27, 2009, herein incorporated by reference in its entirety, discloses methods of making mixed triglycerides using crude glycerol for use as plasticizers for polymer resins.

With regard to the present disclosure, the applicants have surprisingly discovered triglyceride esters made from a mixed acid blend including linear or branched alkyl acids with benzoic acid or toluic acid or a mixture of the two thereof results in a plasticizer with improved hydrolytic stability. Hence, replacing one or more of the linear or branched alkyl groups of the triglyceride ester with an aryl group improves the hydrolytic stability of the plasticizer.

In one form of the present disclosure, the triglyceride plasticizer is “phthalate-free”. As used in the instant specification and in the appended claims, the term “phthalate-free” means that the plasticizer does not contain any phthalate diesters, which are also known in the art simply as phthalates.

Referring to the triglyceride chemical formula below, for the instant application including the claims, the total carbon number of the triester groups is defined as the sum of the carbons for the R¹, R² and R³ groups plus the three carbons for the three carbonyl groups, and not including the three glycerol backbone carbons. Hence for illustrative purposes, for a C₈ triglyceride (also referred to as 888 triglyceride), the total carbon number would be 24 as defined herein (7+7+7=21 carbons from the R¹, R², and R³ alkyl groups plus three carbonyl group carbons) because the three glycerol backbone carbons are not included in the calculation. For a C₇ triglyceride (also referred to as 777 triglyceride), the total carbon number would be 21 as defined herein (6+6+6=18 carbons from the R¹, R², and R³ alkyl groups plus three carbonyl group carbons) because the three glycerol backbone carbons again are not included in the calculation. When R is a benzoate group, seven carbons are added to the total carbon number for the triglyceride. When R is a toluate group, eight carbons are added to the total carbon number for the triglyceride. 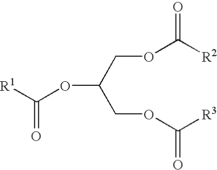
According to the present disclosure, the triglycerides disclosed herein may be produced by esterification of a mixed acid blend comprising one or more C₄ to C₁₃ linear or branched acids with benzoic acid, toluic acid or a combination thereof at a molar ratio of 1:1 or ranging from 0.25:1 to 4:1, or 0.33:1 to 3:1, or 0.5:1 to 2:1, or 0.67:1 to 1.5:1. The molar ratios above of the one or more C₄ to C₁₃ linear or branched acids with benzoic acid, toluic acid or a combination thereof will yield a triglyceride having a molar ratio of C₃ to C₁₂ linear or branched alkyl groups to benzoate and/or toluate groups in the triglyceride ranging from 0.5:1 to 2:1, or 1:1 to 2:1, or 1.4:1 to 2:1, or 0.5:1 to 1.4:1, or 0.5:1 to 1:1 with a total carbon number of the linear or branched alkyl triester groups ranging from 20 to 25 (including the three carbons for the three carbonyl groups and not including the three glycerol backbone carbons).

In one embodiment, the at least one or more C₄ to C₁₃ linear or branched acids will be derived from the hydroformylation of light olefins, aldol condensation of the light aldehydes and then hydrogenation followed by oxidation and thus may be referred to herein as “oxo acids”. The OXO Process is per se well known. By way of recent examples, see, for instance, U.S. Pat. Nos. 7,345,212; 7,186,874; 7,148,388; 7,081,554; 7,081,553; 6,982,295; 6,969,736; 6,969,735; 6,013,851; 5,877,358; and PCT publications WO2007106215; WO2007040812; WO2006086067; WO2006055106; WO2003050070; WO2000015190. However, it will be recognized by one of skill in the art that the C₄ to C₁₃ linear or branched acids may be derived from other processes. In another embodiment, the one or more C₄ to C₁₃ branched acids may be Neo acids.

In some embodiments of the invention, the oxo-acids used to esterify the glycerol have an average branching of from about 0.2 to about 4.0 branches per molecule, preferably from about 0.8 to about 3.0 branches per molecule. In one embodiment, the average branching may range from about 1.0 to about 2.4 branches per molecule. In another embodiment, C₅ to C₈ acids are used having an average branching of from about 1.2 to about 2.2 branches per molecule, preferably from about 1.2 to about 2.0, more preferably from about 1.2 to about 1.8 branches per molecule. In other embodiments, the average branching per molecule of the oxo-acids used to esterify the glycerol will be from about 1.2 to about 1.6. In yet other embodiments, the oxo-acids used may have the branching properties of their precursor olefins described in International Patent Applications WO03/082778 and WO03/082781, U.S. Patent Application US2005/0014630, or U.S. Pat. No. 7,507,868, all herein incorporated by reference.

Nuclear Magnetic Resonance analyses of the branching found in the oxo-acids finds that these branches are typically methyl groups. For example, with the branched C₇ oxo-acid, typical isomers include 2-methylhexanoic acid, 3-methylhexanoic acid, 4-methylhexanoic acid, and 5-methylhexanoic acid, as well as some 3,4-dimethylpentanoic acid, 2,4-dimethylpentanoic acid, 2,2-dimethylpentanoic acid, 3,3-dimethylpentanoic acid, 2,3-dimethylpentanoic acid, and 2,3,3-trimethylbutanoic acid. Some n-heptanoic acid, 2-ethylpentanoic acid, and 3-ethylpentanoic acid are also present. Similar products are found with mixtures of isomers in the C₈ and C₉ oxo-acids. C₉ oxo-acids, when prepared from the Oxo reaction using diisobutylene as the olefin feed, will give mostly trimethyl branched acids, such a 3,5,5-trimethylhexanoic acid. The oxo-acids generally provide more than one isomer. Table 1 provides typical branching characteristics of C₄-C₁₃ oxo-acids.

TABLE 1
¹³C NMR Branching Characteristics of Typical OXO-Acids.

| | Aver- | | | | % Carbon- |
|---|---|---|---|---|---|
| | age Car- | Pendant | Total | Pendant | yls α to |
| OXO-Acid | bon No. | Methyls^{a} | Methyls^{b} | Ethyls | Branch |
| C₄^{c} | 4.0 | 0.35 | 1.35 | 0 | 35 |
| C₅^{d} | 5.0 | 0.35 | 1.35 | 0 | 30 |
| C₆ | — | — | — | — | — |
| C₇ | 6.88-7.92 | 0.98-1.27 | 1.94-2.48 | 0.16-0.26 | 11.3-16.4 |
| C₈ | 8.1-8.3 | — | 2.7 | — | 12-15 |
| C₉ | 9.4 | — | n/a | — | 12 |
| C₁₀ | 10.2 | — | n/a | — | 12 |
| C₁₂ | — | — | — | — | — |
| C₁₃ | 12.5 | — | 4.4 | — | 11 |

—Data not available.
^{a}C₁ Branches only.
^{b}Includes methyls on all branch lengths and chain end methyls.
^{c}Calculated values based on an assumed molar isomeric distribution of 65% n-butanoic acid and 35% isobutanoic acid (2-methylpentanoic acid).
^{d}Calculated values based on an assumed molar isomeric distribution of 65% n-pentanoic acid, 30% 2-methylbutanoic acid, and 5% 3-methylbutanoic acid.

The present disclosure is also directed to the product of the process, which comprises at least one compound according to the following structure (I): 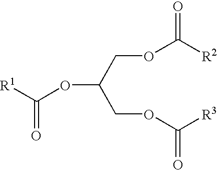
wherein the sum of the carbons for the linear or branched alkyl and aryl triester groups (R¹, R², and R³ plus three carbons for the three carbonyl groups and not including the three glycerol backbone carbons) may range from 20 to 25, and wherein the aryl groups are selected from benzoate groups, toluate groups and combinations thereof, and wherein the molar ratio of C₃ to C₁₂ linear or branched alkyl groups to benzoate and/or toluate groups in the triglyceride ranges from 0.5:1 to 2:1. In one embodiment, the product of the above mentioned process comprises at least 45 wt %, or at least 55 wt %, or at least 65 wt %, or at least 70 wt %, or at least 75 wt %, or at least 85 wt %, or at least 95 wt %, or at least 99 wt %, or 100 wt % of the plasticizer composition. Alternatively, the sum of the carbons for the linear or branched alkyl and aryl triester groups (R¹, R², and R³ plus three carbons for the three carbonyl groups and not including the three glycerol backbone carbons) may range from 20 to 24, or 20 to 23, or 20 to 22, or 20 to 21, or 22 to 25, or 23 to 25, or 24 to 25. In another form, the sum of the carbons for the R¹, R², and R³ plus three carbons for the three carbonyl groups and not including the three glycerol backbone carbons may be 20, or 21, or 22, or 23, or 24, or 25.

The present disclosure is also directed to the product of the process, which comprises at least one compound according to structure (I), wherein the sum of the carbons for the branched alkyl and aryl triester groups (R¹, R², and R³ plus three carbons for the three carbonyl groups and not including the three glycerol backbone carbons) may range from 20 to 25, and also wherein R¹, R², and R³ are independently selected from C₃ to C₁₂ alkyl groups having an average number of branches of from about 0.2 to about 4.0 branches per group, preferably from 0.8 to about 3.0 branches per group, and benzoate or toluate groups, or combinations thereof. In one embodiment, the average branching may range from about 1.0 to about 2.4 branches per alkyl group. In another embodiment, the alkyl groups are C₄ to C₇ groups having an average branching of from about 1.2 to about 2.2 branches per group, preferably from about 1.2 to about 2.0, more preferably from about 1.2 to about 1.8 branches per group. In other embodiments, the average branching of the alkyl groups will be from about 1.2 to about 1.6 branches per group. In yet other embodiments, the alkyl groups used may have the branching properties of their precursor olefins described in International Patent Applications WO03/082778 and WO03/082781, U.S. Patent Application US2005/0014630, or U.S. Pat. No. 7,507,868, all herein incorporated by reference.

In yet another embodiment, the blend of triglycerides may include tribenzoate or other triaryl triglycerides, wherein the total of the tribenzoate or other triaryl triglycerides is less than 55 wt % of the blend. In an alternate embodiment, the triaryl triglycerides are absent from the mixture, having been removed via distillation and recycled back for transesterfication with alkyl oxo-acids.

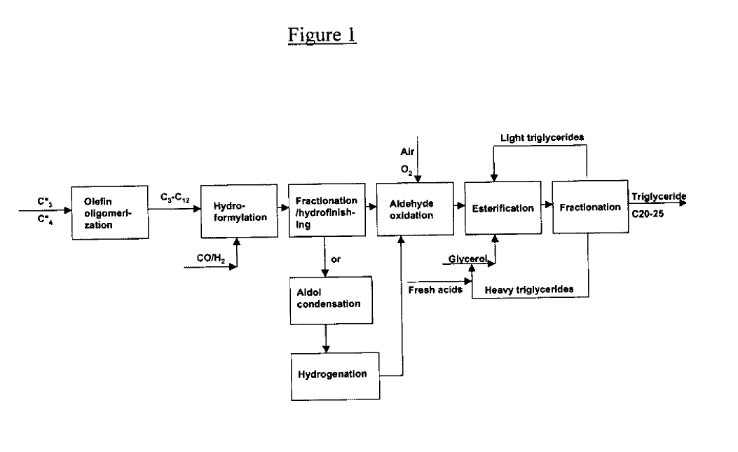

In the first step of the process for producing triglycerides disclosed herein, linear or branched aldehydes may be produced by hydroformylation of C₃ to C₁₂ olefins that in turn have been produced by propylene, butene, and/or pentene oligomerization over solid phosphoric acid or zeolite catalysts. The oligomerization processes are per se well-known. See, for instance, U.S. Pat. Nos. 7,253,330, and 7,145,049. The hydroformylation process step is depicted in FIG. 1. The hydroformylation process produces a mixture of aldehydes and alcohols depending upon the catalyst used and the processing conditions. In one form, the hydroformylation reaction may be catalyzed by a metal selected from Groups 8-10 according to the new notation for the Periodic Table as set forth in Chemical Engineering News, 63(5), 27 (1985). In particular, Rh catalysts tend to be more selective toward forming aldehydes as opposed to alcohols compared to Co catalysts. The non-limiting exemplary metal catalysts selected from Rh and Co may also be used with an organic ligand to further improve catalyst activity and selectivity. In another form, the feed for the hydroformylation process may be formed by dimerizing a feedstock selected from propylene, butenes, pentenes and mixtures thereof by solid phosphoric acid or a zeolite dimerization.

In one form, the resulting C₄ to C₁₃ aldehydes can then be recovered from the crude hydroformylation product stream by fractionation as depicted in FIG. 1 to remove unreacted olefins and the corresponding alcohols. These C₄ to C₁₃ aldehydes can then in turn be oxidized to their respective C₄ to C₁₃ acids using air or enriched air as an oxygen source as depicted in FIG. 1. In an alternative form, that avoids the previous fractionation step, the one or more C₄ to C₁₃ linear or branched alkyl aldehydes/alcohols can be oxidized to the corresponding acids and alcohols and then the unreacted aldehydes purified by distillation. The separated unreacted aldehydes plus the alcohols are oxidized to their corresponding acids. This alternative form may be particularly suitable when using a Rh catalyst during the hydroformylation process. In either of the preceding forms, the distilled aldehydes may be oxidized to an acid followed by fractionation to remove unreacted alcohol. The oxidizing steps may be either catalyzed or non-catalyzed.

Non-limiting exemplary C₄ to C₁₃ acids include acetic acid, bromoacetic acid, propanoic acid, 2-chloropropanoic acid, 3-chloropropanoic acid, 2-methylpropanoic acid, 2-ethylpropanoic acid, 2-methylbutanoic acid, 3-methylbutanoic acid, 2-ethylbutanoic acid, 2,2-dimethylbutanoic acid, 2,3-dimethylbutanoic acid, 3,3-dimethylbutanoic acid, 2-methylpentanoic acid, 3-methylpentanoic acid, 4-methylpentanoic acid, cyclopentyl acetic acid, cyclopentyl propanoic acid, cyclopentyl hexanoic acid, cyclohexane carboxylic acid, cyclohexane acetic acid, 2-ethylhexanoic acid, nonadecafluorodecanoic acid, decanoic acid, and undecanoic acid.

Following the oxidation reaction, the C₄ to C₁₃ acids can then be purified by fractionation to remove unreacted aldehydes, lights and heavies formed during oxidation.

The next step in the process includes combining the linear, branched or combination thereof C₄ to C₁₃ acid at a molar ratio of from 0.25:1 to 4:1 with benzoic acid, toluic acid or combinations thereof to form a mixed acid blend. When a blend of benzoic acid and toluic acid is used, the weight percent of benzoic acid may be from 10 wt % to 90 wt % with the remainder being toluic acid. For example, the benzoic acid may be 10, 20, 30, 40, 50, 60, 70, 80, or 90 wt % of the blend with the remainder being toluic acid. The molar ratio of C₄ to C₁₃ acid to benzoate or toluate in the mixed acid blend may be 1:1 or may range from 0.25:1 to 4:1, or 0.33:1 to 3:1, or 0.5:1 to 2:1, or 0.67:1 to 1.5:1. The toluic acid may include the ortho isomer, the meta isomer, the para isomer, and combinations thereof. The C₄ to C₁₃ acid may be a linear acid or a branched acid, with exemplary non-limiting branched acids including Oxo acids and Neo acids.

The next step in the process, as depicted in FIG. 1, is the esterification of the mixed acid blend with glycerol to form a triglyceride. Alternatively, other polyols may be used to esterify the mixed acid blend. Such polyols may have two alcohol groups, or three (as for glycerol), or four, or other quantity of multiple alcohol groups. Other non-limiting exemplary polyols include ethylene glycol, poly(ethylene glycol), propylene glycol, poly(propylene glycol), triethylene glycol, and triethylene glycol derivatives, as well as dimers of ethylene glycol and/or propylene glycol and other C₂ to C₆ diols or glycols. When polyols other than glycerol are used to esterify the mixed acid blend, a mixed alkyl-aryl polyol ester is formed rather than a triglyceride. Mixtures of polyols may be used, such as a mixture of glycerol with propylene glycol, or a mixture of glycerol with triethylene glycol or a triethylene glycol derivative.

Glycerol is currently an attractive polyol for use to make plasticizers because it is abundantly available. It is, for instance, a major byproduct of biodiesel production. When glycerol is used, this process yields a linear alkyl-aryl triglyceride, a branched alkyl-aryl triglyceride, or a combination thereof. The mixed acid blend can then be esterified as depicted in FIG. 1 with glycerol. The esterification step may be catalyzed by at least one metal selected from Ti, Zr or Sn, or a mixture thereof, or catalyzed by an organic acid. In an alternative form, the esterification step may be uncatalyzed. The esterification process used to produce mixed triglycerides with the mixed acid blend including benzoic acid, toluic acid or combinations thereof disclosed herein results in mixed triglycerides with product selectivities comparable to that of using only linear or branched alkyl acids.

Crude glycerol may also be used. The term “crude glycerol” means a glycerol component including not more than 90 wt % of glycerol. Other components may include, but are not limited to, methanol, water, fatty acid, MONG (Matter Organic Not Glycerol), NaCl, ash and/or other impurities. In other forms, the crude glycerol may include not more than 95 wt %, or 90 wt %, or 88 wt %, or 86 wt %, or 84 wt %, or 82 wt %, or 50 wt % glycerol. The inorganic impurities are precipitated at the end of the esterification, and are removed by filtration and washing the ester with water. In other words, the esterification reaction is a means of purifying the crude glycerol. Non-limiting exemplary crude glycerols include REG, EIS-739, EIS-740, EIS-733, EIS-724, EIS 56-81-5, IRE and mixtures thereof.

In another form of the present disclosure, a mixture of crude glycerol with another polyol may be utilized to produce mixtures of triglycerides and other polyol esters that may be used as plasticizers. Other polyols that may be utilized with crude glycerol during the esterification process include, but are not limited to, ethylene glycol, poly(ethylene glycol), propylene glycol, poly(propylene glycol), triethylene glycol and triethylene glycol derivatives, dimers of ethylene glycol and/or propylene glycol, and other C₂ to C₆ diols or glycols Mixtures of crude glycerol with these polyols, such as ethylene glycol, propylene glycol, and/or triethylene glycol, may include at least 20 wt %, or least 40 wt %, or least 60 wt %, or least 80 wt % crude glycerol with the remainder constituting the other polyol. It is preferred that the polyols as part of the crude glycerol or mixtures of crude glycerol with other polyols be fully esterified so that there are a low to negligible amount of free hydroxyl groups. Thus, for example, it is preferred that the glycerol component of the crude glycerol is esterified to the triester.

Single carbon number linear or branched acids can be used in the esterification, or linear or branched acids of differing carbon numbers can be used in the mixed acid blend with benzoic or toluic acid to optimize product cost and performance requirements. Hence, the mixed acid blend may be esterifed to form mixed triglycerides including linear or branched alkyl or aryl esters, wherein the total carbons for the triester groups (R¹, R², and R³ plus three carbons for the three carbonyl groups and not including the three glycerol backbone carbons) ranges from 20 to 25. Such range of total carbons for the triester groups yield triglycerides with outstanding performance when used as plasticizers for polymeric resins. More particularly, triglycerides with linear or branched alkyl and aryl groups with a total carbon number of the triester groups ranging from 20 to 25 have been discovered to yield low volatility and excellent compatibility with a broad range of polymeric resins, including PVC. Such triglycerides also yield outstanding low temperature performance properties.

In particular, it has been found that replacing one or more of the alkyl groups of the triester with an aryl group significantly improves the hydrolytic stability of the resulting plasticizer. In one form, one of the alkyl groups of the triester is replaced with an aryl group. In another form, two of the alkyl groups of the triester are replaced with an aryl group. In the case of single substitution of an alkyl group with an aryl group on the triester, the aryl group may be a benzoate group or a toluate group. In the case where two of the alkyl groups of the triester are substituted with aryl groups, the aryl groups may be two benzoate groups, two toluate groups or one benzoate group and one toluate group. The inclusion of a benzoate or toluate group in the triester improves the chemical stability of the plasticizer towards hydrolysis. These mixed triesters including a combination of two linear or branched alkyl groups and one aryl group, and/or one linear or branched alkyl group and two aryl groups, are also compatible with a wide range of polymers including vinyl-based polymers.

The next step in the process is purifying the linear, branched or combination thereof alkyl-aryl triglyceride to form a plasticizer, wherein the total carbon number of the triester groups (aryl and alkyl) ranges from 20 to 25 and includes from one to two aryl groups for greater than or equal to 45 wt % of the plasticizer.

Following the esterification process, a fractionation process, such as distillation, may be used to separate the C₂₀ to C₂₅ triglycerides from the lighter and heavier triglycerides. The light triglycerides may be recycled back to the esterification step of the process, to undergo transesterification into the desired C₂₀ to C₂₅ triglycerides. The heavy triglycerides may also be recycled back to the esterification step of the process after adding fresh acids and glycerol. The C₂₀ to C₂₅ triglycerides may be triglycerides containing C₄ to C₁₃ acid groups and one or more aryl groups, given that in at least 45 wt %, or at least 70 wt %, or at least 95 wt % of the species, includes one or two of the groups on the triester, which is either a benzoate group or a toluate group, or a combination thereof, and the triester groups have a carbon number ranging from 20 to 25 in at least 45 wt % of the plasticizer. Note, however that these C₂₀ to C₂₅ triglycerides may include other proportions (55 wt % or less relative to the total) of triglycerides which do not have a total carbon number of the triester groups falling within the 20 to 25 range, and/or triester groups of the triglycerides not including one or two benzyl or toluate groups. If the total weight % of these non-inventive, non-C₂₀ to C₂₅ triglycerides is greater than 55 wt %, plasticizer properties (volatility, compatibility, low temperature performance, etc.) will begin to be negatively impacted. Hence, for the C₂₀ to C₂₅ triglycerides disclosed herein, linear or branched alkyl and aryl triglycerides with a total carbon number of from 20 to 25 should comprise greater than or equal to 45 wt %, or greater than or equal to 50 wt %, or greater than or equal to 55 wt %, or greater than or equal to 60 wt %, or greater than or equal to 65 wt %, or greater than or equal to 70 wt %, or greater than or equal to 75 wt %, or greater than or equal to 90 wt %, or greater than or equal to 95 wt %, or greater than or equal to 97 wt %, or greater than or equal to 99 wt %, or greater than or equal to 99.5 wt %, or greater than or equal to 99.9 wt % of the plasticizer. The fractionation process following the esterification step may be used to increase the purity of C₂₀ to C₂₅ triglycerides.

Similarly, if the total weight % of non-inventive, all-alkyl or all-aryl triglycerides (with zero or three aryl groups instead of one or two) is greater than 55 wt %, other specific properties such as hydrolytic stability, or the property balance between hydrolytic stability and other parameters, will begin to be negatively impacted. Hence, for the species disclosed herein, those comprising one or two benzoate, toluate, or combination thereof groups on the triester should comprise greater than or equal to 45 wt %, or greater than or equal to 50 wt %, or greater than or equal to 55 wt %, or greater than or equal to 60 wt %, or greater than or equal to 65 wt %, or greater than or equal to 70 wt %, or greater than or equal to 75 wt %, or greater than or equal to 90 wt %, or greater than or equal to 95 wt %, or greater than or equal to 97 wt %, or greater than or equal to 99 wt %, or greater than or equal to 99.5 wt %, or greater than or equal to 99.9 wt % of the plasticizer. The fractionation process following the esterification step may be used to increase the purity of mono- or di-aryl triglycerides.

The chemistry and a simplified process to produce triglycerides via the route described above is shown in equations (1)-(4), below. For simplicity, a branched hexene feed is shown as a representative olefin in equation (1), but the olefin feed can be linear or branched propene, butenes, pentenes, hexenes, heptenes, octenes, nonenes, decenes, undecenes, or dodecenes as the starting olefins. As discussed above, the resulting C₄, C₅, C₆, C₇, C₈, C₉, C₁₀, C₁₁, C₁₂, and C₁₃ acids are used in combination with benzoic, toluic acid or a mixture of the two to make mixed carbon number esters to be used as plasticizers, as long as the sum of carbons for the triester groups (R¹, R², and R³ plus three carbons for the three carbonyl groups and not including the three glycerol backbone carbons) for greater than or equal to 45 wt % of the plasticizer product is from 20 to 25 and the amount of plasticizer product having from 1 to 2 aryl groups is greater than or equal to 45 wt %. Equation (3) shows the preparation of benzoic acid by oxidation of toluene. Correspondingly, the C₄-C₁₃ acids may be linear, branched, or a combination thereof. The linear or branched C₄-C₁₃ acid is combined with either benzoic acid, toluic acid or a mixture of the two to form an alkyl-aryl mixed acid blend for the esterification step of the process. This mixing of carbon numbers and levels of branching with the inclusion of aryl group(s) from either benzoic acid, toluic acid or a combination of the two may be manipulated to achieve the desired compatibility with PVC for the respective polyol used for the polar end of the plasticizer, and to meet other plasticizer performance properties, such as hydrolytic stability. Equation 4 below would necessarily include either toluic acid, benzoic acid or a mixture of the two as part of a mixed acid blend for the esterification step.

Benzoic acid is made from toluene oxidation using conventional processes. Toluic acid is made from xylene oxidation using conventional processes. Toluic acid may include the ortho isomer, the meta isomer, the para isomer, and combinations thereof. After esterification, the benzoate group adds seven carbons to the mixed triglyceride. In contrast, after esterification, the toluate group adds eight carbons to the mixed triglyceride.

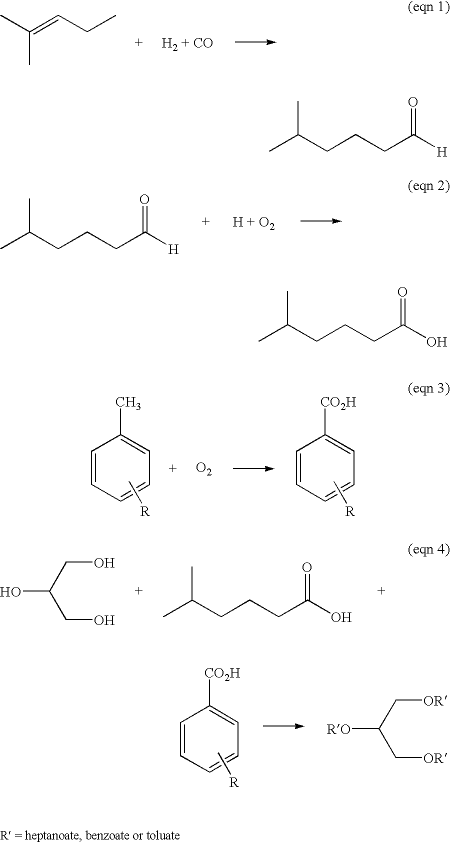

Equations 5-8 describe a second route for the formation of acids via hydroformylation followed by aldol condensation, hydrogenation, then oxidation:

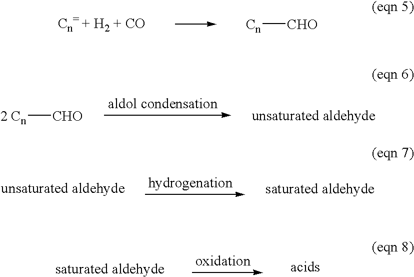

The applicability of the triglyceride structures as potential PVC plasticizers can be screened by estimating their relative solubility in PVC using Small's group contribution method to calculate solubility parameters for each structure (see: (a) The Technology of Plasticizers by J. Sears and J. Darbey, John Wiley & Sons, New York, 1982, pp 95-99, discussing use of Small's formula to predict plasticizer compatibility with PVC; (b) Small, P. A., “Some Factors Affecting the Solubility of Polymers”, J. Appl. Chem, 3, pp 76-80 (1953) which cites Small's original work as a reference; (c) Polymer Handbook, 3rd Ed., J. Brandrup & E. H. Immergut, Eds. John Wiley, New York, (1989), which includes use of Small's group contribution values). It is noted that solubility parameter data alone does not predict other critical performance factors, such as volatility, in addition to compatibility with PVC. These calculations are shown below in Table 2 for diisononyl phthalate (DINP) as a reference (MW=molecular weight):

TABLE 2

| | | Number | Solubility | | MW |
|---|---|---|---|---|---|
| DINP | Polarity | of Groups | Contribution | MW | Contribution |
| CH₃ | 214 | 2 | 428 | 15 | 30 |
| —CH₂— | 133 | 16 | 2128 | 14 | 224 |
| COO esters | 310 | 2 | 620 | 44 | 88 |
| Phenylene | 658 | 1 | 658 | 76 | 76 |
| | | | 3834 | | 418 |

Solubility Parameter = 8.878737
Density = 0.968
Delta to PVC = −0.78126

Likewise, the solubility may also be calculated for the mixed benzoate and aliphatic triglyceride esters. Tables 3-6 show solubility parameter calculations for the possible triglycerides made from glycerol esterification with benzoic (Ben) and C₇ acids (C7).

TABLE 3

| | | Number | Solubility | | MW |
|---|---|---|---|---|---|
| C7C7C7 | Polarity | of Groups | Contribution | MW | Contribution |
| CH₃ | 214 | 6 | 1264 | 15 | 90 |
| —CH₂— | 133 | 11 | 1463 | 14 | 154 |
| —CH═ | 28 | 4 | 112 | 13 | 52 |
| COO esters | 310 | 3 | 930 | 44 | 132 |
| | | | 3789 | | 428 |

Solubility Parameter = 8.50
Density = 0.96
Delta to PVC = −1.16

The C₇ triglyceride (also referred to as 777 triglyceride) composition with a total carbon number of 21 (excluding the three glycerol backbone carbons) yields adequate volatility and excellent compatibility when used in PVC resins as a plasticizer.

TABLE 4

| | | Number | Solubility | | MW |
|---|---|---|---|---|---|
| BenC7C7 | Polarity | of Groups | Contribution | MW | Contribution |
| CH₃ | 214 | 2 | 428 | 15 | 30 |
| —CH₂— | 133 | 10 | 1330 | 14 | 140 |
| COO esters | 310 | 3 | 930 | 44 | 132 |
| Phenylene | 658 | 1 | 658 | 76 | 76 |
| | | | 3346 | | 378 |

Solubility Parameter = 8.568593
Density = 0.968
Delta to PVC = −1.09141

TABLE 5

| | | Number | Solubility | | MW |
|---|---|---|---|---|---|
| BenBenC7 | Polarity | of Groups | Contribution | MW | Contribution |
| CH₃ | 214 | 1 | 214 | 15 | 15 |
| —CH₂— | 133 | 5 | 665 | 14 | 70 |
| COO esters | 310 | 3 | 930 | 44 | 132 |
| Phenylene | 658 | 2 | 1316 | 76 | 152 |
| | | | 3125 | | 369 |

Solubility Parameter = 8.197832
Density = 0.968
Delta to PVC = −1.46217

TABLE 6

| | | Number | Solubility | | MW |
|---|---|---|---|---|---|
| BenBenBen | Polarity | of Groups | Contribution | MW | Contribution |
| CH₃ | 214 | 0 | 0 | 15 | 0 |
| —CH₂— | 133 | 0 | 0 | 14 | 0 |
| COO esters | 310 | 3 | 930 | 44 | 132 |
| Phenylene | 658 | 3 | 1974 | 76 | 228 |
| | | | 2904 | | 360 |

Solubility Parameter = 7.808533
Density = 0.968
Delta to PVC = −1.85147

The solubility parameter of PVC is calculated by the same Small's Group Contribution Method to be 9.66. The differences in solubility parameters between the triglyceride structures and PVC are shown in Tables 2-6. These differences from PVC range from 1.16 for the C₇ triglyceride (also referred to as 777 triglyceride) to 1.85 units for the benzoate triglyceride (also referred to as BenBenBen triglyceride), which indicates reasonable expected solubility in PVC for these materials. As references, the solubility parameter for DINP is 8.88 (delta to PVC=0.78) (Table 2). The estimated solubility parameter for the non-phthalate plasticizer di-isononyl cyclohexanoate is 7.32 by Small's method. This is a difference of 2.34 solubility parameter units from PVC.

A non-limiting process embodiment is illustrated in FIG. 1. Propylene and butene are used as feedstock for an oligomerization reaction. The reaction may be continuous, batch, or semibatch. Unreacted C₃/C₄ olefins are distilled off and optionally recycled. Or, C₃-C₁₂ olefins (monomers, dimers, trimers and/or tetramers) are provided to the hydroformylation reaction to form C₄-C₁₃ aldehydes and other by-products. Carbon monoxide and hydrogen, conveniently supplied as Syngas, are also supplied to the reactor. The products are then separated by fractionation, with olefins optionally recycled and the aldehydes and alcohols being separated. The amount of aldehyde and alcohols produced may be attenuated in the hydrofinishing section. In an embodiment, the aldehydes are then oxidized with the addition of air and/or oxygen, and unreacted aldehydes and heavies are separated out. The desired product Cₙ (n=4-13) acid in combination with benzoic acid, toluic acid or combinations thereof, is then esterified with a polyol, in this embodiment glycerol, and recovered as a triglyceride wherein the total carbon number (excluding the three glycerol backbone carbons) of the triester groups ranges from 20 to 25 and includes from one to two aryl groups for greater than or equal to 45 wt % of the triglyceride.

In another form of the present disclosure, a composition comprising a blend of two or more different triglycerides may also provide outstanding plasticizer performance in a range of polymer resins, including PVC. The blend of the two or more different triglycerides should include triglycerides according to the composition and process of making disclosed herein. That is, at least 45 wt % of the individual triglycerides in the mixed triglyceride blend are linear or branched alkyl-aryl triglyceride wherein the total carbon number of the triester groups ranges from 20 to 25. Furthermore, in at least 45 wt % of the individual triglycerides in the mixed triglyceride blend, two of the triester groups are linear and/or branched alkyl groups (Cₙ) and one of the triester groups is an aryl group (benzoate (Ben) or toluate or combinations thereof); or, one of the triester groups is linear and/or branched alkyl groups and two of the triester groups are an aryl group (benzoate or toluate or combinations thereof). In one embodiment, the mixed triglyceride includes a two-component blend of triglycerides having the structures CₙBenCₙ/CₙCₙBen and BenBenCₙ/BenCₙBen (where the sidechain groups are listed to indicate in order of position on the triglyceride backbone; e.g. “CₙBenCₙ”=a triglyceride with Cₙ groups at the external (1 and 3) ester positions and a Ben group at the internal (2) ester position). Note, however that these mixed triglyceride blends may include other proportions (defined herein as 55 wt % or less relative to the total) of triglycerides which do not have a total carbon number of the triester groups falling within the 20 to 25 range and/or triglycerides which do not include two alkyl groups and one aryl group or two aryl groups and one alkyl group on the triester. If the total weight % of these non-inventive triglycerides is greater than 55 wt % in the mixed triglyceride blend, plasticizer properties (volatility, compatibility, low temperature performance, hydrolytic stability, etc.) will begin to be negatively impacted. Hence, for the mixed triglycerides disclosed herein, linear or branched alkyl-aryl triglycerides with a total carbon number (excluding the three glycerol backbone carbons) of from 20 to 25 should comprise greater than or equal to 45 wt %, or greater than or equal to 50 wt %, or greater than or equal to 55 wt %, or greater than or equal to 60 wt %, or greater than or equal to 65 wt %, or greater than or equal to 70 wt %, or greater than or equal to 75 wt %, or greater than or equal to 90 wt %, or greater than or equal to 95 wt %, or greater than or equal to 97 wt %, or greater than or equal to 99 wt %, or greater than or equal to 99.5 wt %, or greater than or equal to 99.9 wt % of the mixed triglyceride blend. Furthermore, those comprising one or two benzoate, toluate, or combination thereof groups on the triester should comprise greater than or equal to 45 wt %, or greater than or equal to 50 wt %, or greater than or equal to 55 wt %, or greater than or equal to 60 wt %, or greater than or equal to 65 wt %, or greater than or equal to 70 wt %, or greater than or equal to 75 wt %, or greater than or equal to 90 wt %, or greater than or equal to 95 wt %, or greater than or equal to 97 wt %, or greater than or equal to 99 wt %, or greater than or equal to 99.5 wt %, or greater than or equal to 99.9 wt % of the mixed triglyceride blend. These mixed triglyceride blends may also be used as plasticizers and yield outstanding properties and performance with a variety of polymer resins. In particular, the hydrolytic stability of resin compositions including the plasticizers disclosed herein are improved due to the presence of one or two aryl groups on the triglyceride.

The plasticizers according to the current disclosure may also be used with polyvinyl chlorides, polyesters, polyurethanes, ethylene-vinyl acetate copolymer, rubbers, acrylics, and polymer blends, such as blends of polyvinyl chloride with an ethylene-vinyl acetate copolymer or polyvinyl chloride with a polyurethane or ethylene-type polymer. In particular, the plasticizers disclosed herein yield improved hydrolytic stability or resistance in resin compositions. Hydrolytic stability is measured via acid and glycerol formation stability in the resin composition. This may be quantified by measuring the hydrolysis products by gas chromatography of resin compositions including the inventive plasticizers disclosed herein. The hydrolysis products of the resin compositions including the plasticizers disclosed herein may be less than 5 wt %, or less than 2 wt %, or less than 1 wt %, or less than 0.5 wt %, or less than 0.4 wt %, or less than 0.3 wt %, or less than 0.2 wt %, or less than 0.1 wt % of the mixture of the resin and plasticizer following melt processing as measured by gas chromatography.

### EXAMPLES

General Procedure for Esterification

Into a four necked 1000 mL round bottom flask equipped with an air stirrer, nitrogen inductor, thermometer, Dean-Stark trap and chilled water cooled condenser were added 0.8 mole glycerol, 1.6 mole acid which has n carbons and could be linear or branched or a mixture thereof, and 1.6 mole benzoic acid, or o-, m-, p-toluic acid or combinations thereof. The Dean-Stark trap was filled with the lighter boiling acid to maintain the same molar ratio of acids in the reaction flask. The reaction mixture was heated to 220° C. with air stirring under a nitrogen sweep. The water collected in the Dean-Stark trap was drained frequently and measured to quantify conversion. The reaction was heated until near-complete or complete conversion was seen; typically ˜10 hours or as indicated in the specific Examples. The products were purified, characterized, and/or fractionated as described in the specific Examples. Gas chromatography analysis on the products was conducted using a Hewlett-Packard 5890 GC equipped with a HP6890 autosampler, a HP flame-ionization detector, and a J&W Scientific DB-1 30 meter column (0.32 micrometer inner diameter, 1 micron film thickness, 100% dimethylpolysiloxane coating). The initial oven temperature was 60° C.; injector temperature 290° C.; detector temperature 300° C.; the temperature ramp rate from 60 to 300° C. was 10° C./minute with a hold at 300° C. for 14 minutes. The calculated %'s reported for products were obtained from peak area, with an FID detector uncorrected for response factors. Composition for the triglyceride products is given in the following manner: a mixed triglyceride of acids with X and Y carbon numbers, wherein X is a linear or branched alkyl group of carbon number X, and Z is an aryl group (benzoate (Ben) or toluate (Tol)), may theoretically contain products with two chains of length X and one of length Z (denoted XXZ or XZX), two chains of length Z and one of length X (denoted XZZ or ZXZ), in addition to products containing three chains of length X (XXX) and three aryl chains (ZZZ). In these abbreviations, the first and third characters represent the terminal (primary) glyceride chains and the second character represents the internal (secondary) glyceride chain. The sum of the carbon numbers for the three groups (including the three carbonyl carbons and not including the three glycerol backbone carbons) ranges from 20 to 25 for at least 45 wt % of the plasticizer product. The weight percent of triglycerides in the mixture having both one or two aryl groups and a carbon number between 20 and 25 is at least 45 wt %.

### Illustrative Example 1

### Synthesis of Mixed Triglyceride of Oxo C₉ Acid and Benzoic Acid

0.8 mole glycerol was esterified with 1.6 mole benzoic acid and 1.6 mole ExxonMobil Chemical Co. Oxo C₉ acids (a mixture of multiple branched and linear isomers) for 10 hours. The Oxo C₉ acid mixture used had the following branching parameters as determined by ¹³C NMR: average carbon number 9.4; 12% of carbonyl groups alpha to a branched alkyl carbon. The unreacted acids were removed by distillation. The mother solution was analyzed by GC (the isomeric distribution or purity could not be determined due to coelution of the isomers), then washed with 2×100 mL of a 10% aqueous sodium carbonate solution. The third and final wash was with 100 mL of distilled water. The product (organic) phase was then dried over magnesium sulfate to remove any water remaining. The dried product phase was then treated with activated charcoal (1.0 wt %) with stirring at room temperature for two hours to remove color. Finally the charcoal was removed by filtration with a Buchner funnel and filter paper to give the product, which was not further purified.

### Illustrative Example 2

### Synthesis of Mixed Triglyceride of 2-Ethylhexanoic Acid and Benzoic Acid

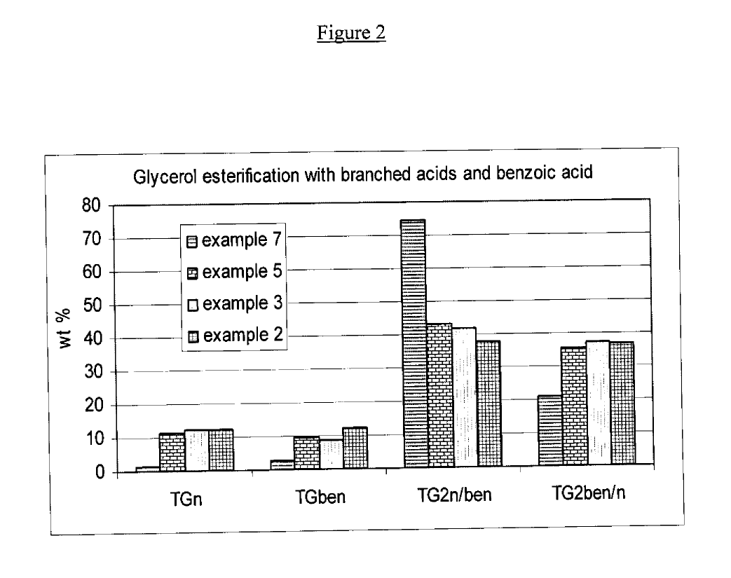

The same procedure as in Illustrative Example 1 was used, substituting 2-ethylhexanoic acid (2EH) for Oxo C₉ acids, except that the total run time was 12 hours. The isomeric distribution of the triglyceride product by GC, as shown in FIG. 2, was as follows: 12.37% 2EH2EH2EH, 12.19% 2EH2EHBen, 25.72% 2EHBen2EH, 23.91% Ben2EHBen, 12.84% BenBen2EH, 12.14% BenBenBen (99.17% triglycerides).

### Illustrative Example 3

### Synthesis of Mixed Triglyceride of 3,5,5-Trimethylhexanoic Acid and Benzoic Acid

The same procedure as in Illustrative Example 1 was used, substituting 3,5,5-trimethylhexanoic acid (Me₃C₉) a single-isomer Oxo C₉ acid derived from the dimerization of isobutylene) for Oxo C₉ acids, except that the total run time was 10 hours. The isomeric distribution of the triglyceride product by GC, as shown in FIG. 2, was as follows: 12.31% Me₃C₉Me₃C₉Me₃C₉, 11.23% Me₃C₉Me₃C₉Ben, 29.24% Me₃C₉BenMe₃C₉, 20.82% BenMe₃C₉Ben, 16.53% BenBenMe₃C₉, 8.55% BenBenBen (98.68% triglycerides).

### Illustrative Example 4

### Synthesis of Mixed Triglyceride of Oxo C₇ Acid and Benzoic Acid

The same procedure as in Illustrative Example 1 was used, substituting an ExxonMobil Chemical Co. Oxo C₇ acid (isomeric mixture) for Oxo C₉ acids, except that the total run time was 10 hours. The Oxo C₇ acid mixture used had the following branching parameters as determined by ¹³C NMR: average carbon number 6.88; 0.98 pendant methyls per molecule; 1.94 total methyls per molecule; 0.22 pendant ethyls per molecule; 11.34% of carbonyl groups alpha to a branched alkyl carbon. The isomeric distribution of the triglyceride product by GC was as follows: 10.95% 777, 39.69% 77Ben/7Ben7, 39.88% 7BenBen/Ben7Ben, 9.21% BenBenBen (99.73% triglycerides).

### Illustrative Example 5

### Synthesis of Mixed Triglyceride of Oxo C₇ Acid and Benzoic Acid

Into a four-necked, one liter round bottom flask equipped with a nitrogen inductor, air stirrer, thermometer, and Dean-Stark trap were added the following: glycerol (184.1 g, 2.0 mole), benzoic acid (488.5 g, 4.0 mole) and ExxonMobil Chemical Co. Oxo C₇ acid (isomeric mixture) (521.2 g, 4.0 mole). The Oxo C₇ acid mixture used had the following branching parameters as determined by ¹³C NMR: average carbon number 7.92; 1.27 pendant methyls per molecule; 2.48 total methyls per molecule; 0.26 pendant ethyls per molecule; 16.39% of carbonyl groups alpha to a branched alkyl carbon. The contents of the flask were heated from 171 to 220° C. for a total of 13 hours; 96.3% conversion was observed in five hours by water removal. The excess acids were removed by distillation under high vacuum. The crude residual product was washed with 2×200 mL of a 10% aqueous sodium carbonate solution followed by 200 mL of distilled water. The organic phase was dried over 5 wt % sodium sulfate, filtered, then fractionated by distillation under high vacuum. The late distillate fractions were combined, distilling at 179-226° C./0.1 mm vacuum. The isomeric distribution of the triglyceride product by GC, as shown in FIG. 2, was approximately as follows: 11.17% 777, 43.20% 77Ben/7Ben7, 35.56% 7BenBen/Ben7Ben, 9.42% BenBenBen (99.35% triglycerides with 0.46% diglycerides).

### Illustrative Example 6

### Synthesis of Mixed Triglyceride of C₆ Acid Mixture and Benzoic Acid (1:1 Acid Ratio)

A procedure similar to Example 5 was performed using the following reactants: glycerol (69.07 g, 0.75 mole), benzoic acid (274.8 g, 2.25 mole), hexanoic acid (169.9 g, 1.463 mole) and 2-methylvaleric acid (91.5 g, 0.788 mole). The reaction mixture was heated for a total of nine hours at 153-220° C. 92.3% conversion was observed after five hours heating by water removal. The excess acids were removed by distillation under high vacuum. The crude products were first treated with decolorizing charcoal (1.0 wt %) with stirring at room temperature for two hours then filtered twice. Next the crude products were washed with aqueous saturated sodium carbonate (10 wt %) followed by distilled water, then dried over sodium sulfate. The crude residual product was not distilled. The isomeric distribution of the triglyceride product by GC was as follows: 10.7% 666, 41.1% 66Ben/6Ben6, 38.9% 6BenBen/Ben6Ben, 8.5% BenBenBen (99.2% triglyceride).

The following two Examples (7-8) demonstrate how the product distributions and properties of the mixed alkyl-aryl triglyceride esters can be easily manipulated from that of Example 6, without need for complicated fractionation of the products. In these two examples, the content of C₂₀-C₂₅ triglycerides is not above 45 wt %, but similar modifications applied to Example 5 (using a C₇ rather than a C₆ acid) would provide such materials.

### Illustrative Example 7

### Synthesis of Mixed Triglyceride of C₆ Acid Mixture and Benzoic Acid (2:1 Acid Ratio)

A procedure similar to Example 5 was performed using the following reactants: glycerol (69.07 g, 0.75 mole), benzoic acid (183.2 g, 1.5 mole), hexanoic acid (226.5 g, 1.95 mole) and 2-methylvaleric acid (122 g, 1.05 mole). The reaction mixture was heated for a total of eight hours at 187-220° C. 100% conversion was observed after four hours heating by water removal. The excess acids were removed by distillation under high vacuum. The crude products were washed with aqueous saturated sodium carbonate, followed by 200 mL of distilled water then dried over sodium sulfate. The crude residual product was not distilled. The isomeric distribution of the triglyceride product by GC, as shown in FIG. 2, was as follows: 1.41 666, 74.59% 66Ben/6Ben6, 21.1% 6BenBen/Ben6Ben, 2.5% BenBenBen (99.0% triglyceride).

### Illustrative Example 8

### Synthesis of Mixed Triglyceride of C₆ Acid Mixture and Benzoic Acid (6.7:1 Acid Ratio)

A procedure similar to Example 5 was performed using the following reactants: glycerol (65.7 g, 0.713 mole), benzoic acid (87.12 g, 0.713 mole), hexanoic acid (359.4 g, 3.094 mole) and 2-methylvaleric acid (193 g, 1.662 mole). The reaction mixture was heated for a total of six hours at 185-208° C. 100% conversion was observed after five hours heating by water removal. The excess acids were removed by distillation under high vacuum. The crude products were washed with aqueous saturated sodium carbonate, followed by distillation then dried over sodium sulfate. The crude residual product was not distilled. The isomeric distribution of the triglyceride product by GC was as follows: 69.2% 666, 25.6% 66Ben/6Ben6, 4.7% 6BenBen/Ben6Ben, 0.02% BenBenBen (99.52% triglyceride).

### Illustrative Example 9

### Synthesis of Mixed Triglyceride of Neo Oxo C₇ Acid and Benzoic Acid

A procedure similar to Example 5 was performed using the following reactants: glycerol (53.13 g, 0.577 mole), benzoic acid (211.51 g, 1.732 mole), and ExxonMobil Chemical Co. neo-C₇ carboxylic acid (isomeric mixture, 225.5 g, 1.732 mole). The acid mixture comprised the following isomeric balance by GC: 43.0% 2,2-dimethylpentanoic acid, 13.5% 2,2,3-trimethylbutanoic acid, and 37.6% 2-ethyl-2-methylbutanoic acid. It had the following branching parameters as determined by ¹³C NMR: average carbon number 7.24; 1.64 pendant methyls per molecule; 3.23 total methyls per molecule; 0.43 pendant ethyls per molecule; 98% of carbonyl groups alpha to a branched alkyl carbon. The reaction mixture was heated for a total of 15 hours at 210-220° C. 100% conversion was observed after eight hours heating by water removal. The excess acids were removed by distillation under high vacuum. The crude products were treated with decolorizing charcoal (1 wt %) while stirring for two hours at room temperature, then filtered twice. The crude residual product was not distilled. The isomeric distribution of the triglyceride product by GC was as follows: 5.9% 777, 30.1% 77Ben/7Ben7, 42.1% 7BenBen/Ben7Ben, 21.1% BenBenBen (99.3% triglyceride, 0.63% diglyceride).

### Illustrative Example 10

### Synthesis of Mixed Triglyceride of Neo Oxo C₉ Acid and Benzoic Acid

A procedure similar to Example 5 was performed using the following reactants: glycerol (23.02 g, 0.25 mole), benzoic acid (61.06 g, 0.5 mole), and ExxonMobil Chemical Co. neo-C₉ carboxylic acid (isomeric mixture, 158.56 g, 1.0 mole). The neo-C₉ acid mixture used had the following branching parameters as determined by ¹³C NMR: average carbon number 9.17; 4.22 pendant methyls per molecule; 5.4 total methyls per molecule; 100% of carbonyl groups alpha to a branched alkyl carbon. The reaction mixture was heated for a total of 13 hours at 220° C. 90% conversion was observed after five hours heating by water removal. The excess acids were removed by distillation under high vacuum. The crude residual product was not distilled. Of the isomeric product distribution (99.8% triglyceride), only the BenBenBen triglyceride (7.53%) was resolvable by GC.

### Illustrative Example 11

### Hydrolytic Stability Comparison Between Mixed Alkyl-Aryl Triglyceride and Alkyl Triglyceride

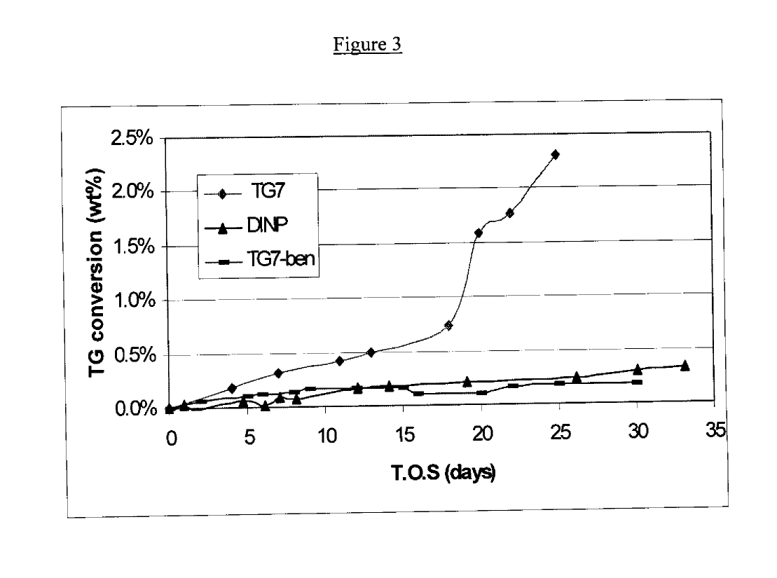

A 120 mL glass Parr reactor was charged with 25 grams of an aqueous 0.05N HCl solution plus 75 grams of either (1) the mixed C₇/benzoic triglyceride ester of Example 5; (2) a comparative alkyl triglyceride having three C₇ chains derived from the same C₇ acid used to prepare the mixed C₇/benzoic triglyceride of Example 5 (100% triglyceride by GC); or (3) the commercial plasticizer diisononyl phthalate (DINP). The mixture was stirred for 30 days at 91-104° C. with GC sampling throughout the heating period to quantify the amount of triglyceride hydrolyzed to diglyceride or other byproducts (“% TG conversion”). FIG. 3 shows the test results for the three materials. The benzoate-containing triglyceride (“TG7-ben”) exhibits a much higher hydrolytic stability than the all-alkyl triglyceride (“TG7”), performing similarly to DINP.

The following two Examples (Example 12 and Comparative Example 1) demonstrate the synthesis of a mixed alkyl-aryl ester of a polyol that is not glycerol.

### Comparative Example C1

### Synthesis of All-Aryl Diester of Triethylene Glycol and Benzoic Acid

A procedure similar to Example 5 was performed using the following reactants: triethylene glycol (87.92 g, 0.5855 mole) in place of glycerol, benzoic acid (214.5 g, 1.76 mole), and m-xylene (39.8 g, 0.375 mole). The reaction mixture was heated for a total of six hours at 196-220° C. 100% conversion was observed after six hours heating by water removal. The excess acids were removed by distillation under high vacuum. The crude residual product was not distilled. The product was the pure benzyl diester except for two small impurities: 0.13% triethylene glycol mono-benzyl ester and 0.325% diethylene glycol di-benzyl ester.

### Illustrative Example 12

### Synthesis of Mixed Diester of Triethylene Glycol and Benzoic Acid/Oxo C₁₀ Acid

A procedure similar to Example 5 was performed using the following reactants: triethylene glycol (225.3 g, 1.5 mole) in place of glycerol, benzoic acid (274.8 g, 2.25 mole), ExxonMobil Chemical Co. Oxo C₁₀ acids (isomer mixture, 386.8 g, 2.25 mole) and m-xylene (29.0 g, 0.273 mole). The reaction mixture was heated for a total of eight hours at 185-220° C. 100% conversion was observed after five hours heating by water removal. The excess acids were removed by distillation under high vacuum. The crude residual product was not distilled. The isomeric distribution of the product by GC was as follows: ˜13.1% triethylene glycol di-benzyl ester and ˜86.9% triethylene glycol di-C₁₀ ester/triethylene glycol mono-C₁₀ mono-benzyl ester (unresolvable).

### Illustrative Example 13

### Differential Scanning Calorimetry (DSC), Viscosity, and Thermogravimetric Analysis (TGA) Property Study of Neat Esters

Thermogravimetric Analysis (TGA) was conducted on the neat plasticizers using a TA Instruments AutoTGA 2950HR instrument (25-600° C., 10° C./min, under 60 cc N₂/min flow through furnace and 40 cc N₂/min flow through balance; sample size 10-20 mg). Table 7 provides a volatility comparison. Differential Scanning Calorimetry (DSC) was also performed on the neat plasticizers, using a TA Instruments 2920 calorimeter fitted with a liquid N₂ cooling accessory. Samples were loaded at room temperature and cooled to about −130° C. at 10° C./min and analyzed on heating to 75° C. at a rate of 10° C./min. Table 7 provides a glass transition (T_{g}) comparison. T_{g}s given in Table 7 are midpoints of the second heats (unless only one heat cycle was performed, in which case the first heat T_{g}, which is typically in very close agreement, is given). Kinematic Viscosity (KV) was measured at 20° C. according to ASTM D-445-20, the disclosure of which is incorporated herein by reference. Comparative data for a common commercial plasticizer (diisononyl phthalate; Jayflex® (DINP), ExxonMobil Chemical Co.) is also included.

TABLE 7
Volatility, Viscosity, and Glass Transition
Properties of Neat Plasticizers.

| | | | | TGA | | |
|---|---|---|---|---|---|---|
| | TGA | TGA | TGA | Wt | | |
| | 1% Wt | 5% Wt | 10% Wt | Loss at | | |
| | Loss | Loss | Loss | 220° C. | DSC T_{g} | KV (20° C., |
| Ex. No. | (° C.) | (° C.) | (° C.) | (%) | (° C.) | mm²/sec) |
| DINP | 184.6 | 215.2 | 228.5 | 6.4 | −79.1 | 96.81 |
| 1 | 202.2 | 233.5 | 247.7 | 2.5 | −58.5 | 263.59 |
| 2 | 187.6 | 216.9 | 230.7 | 5.9 | −60.5 | 226.96 |
| 3 | 199.9 | 233.3 | 248.3 | 2.6 | −48.1 | 370.28 |
| 4 | 192.8 | 226.1 | 241.4 | 3.7 | −61.6 | — |
| 5 | 176.5 | 210.4 | 225.6 | 7.7 | −61.0 | 374.61 |
| 6 | 171.4 | 204.9 | 220.2 | 9.8 | −65.9 | 93.58 |
| 7 | 162.3 | 193.9 | 208.3 | 16.8 | −78.4 | 42.76 |
| 8 | 155.3 | 187.2 | 201.8 | 23.1 | −93.8 | 19.1 |
| 9 | 168.0 | 202.4 | 218.7 | 10.6 | −45.8 | 882.7 |
| 10 | 179.2 | 213.6 | 229.4 | 6.7 | −38.7 | 6931.11 |
| C1 | 200.3 | 239.1 | 255.2 | 2.2 | −55.0 | — |
| 12 | 192.2 | 225.6 | 241.1 | 3.9 | −78.5 | — |

— Data not taken.

### Illustrative Example 14

### General Procedure for Plasticization of Poly(Vinyl Chloride) with Esters

A 5.85 g portion of the ester sample (or comparative commercial plasticizer DINP) was weighed into an Erlenmeyer flask which had previously been rinsed with uninhibited tetrahydrofuran (THF) to remove dust. An 0.82 g portion of a 70:30 by weight solid mixture of powdered Drapex® 6.8 (Crompton Corp.) and Mark® 4716 (Chemtura USA Corp.) stabilizers was added along with a stirbar. The solids were dissolved in 117 mL uninhibited THF. Oxy Vinyls® 240F PVC (11.7 g) was added in powdered form and the contents of the flask were stirred overnight at room temperature until dissolution of the PVC was complete (a PVC solution for preparation of an unplasticized comparative sample was prepared using an identical amount of stabilizer, 100 mL solvent, and 13.5 g PVC). The clear solution was poured evenly into a flat aluminum paint can lid (previously rinsed with inhibitor-free THF to remove dust) of dimensions 7.5″ diameter and 0.5″ depth. The lid was placed into an oven at 60° C. for two hours with a moderate nitrogen purge. The pan was removed from the oven and allowed to cool for a ˜5 min period. The resultant clear film was carefully peeled off of the aluminum, flipped over, and placed back evenly into the pan. The pan was then placed in a vacuum oven at 70° C. overnight to remove residual THF. The dry, flexible, typically almost colorless film was carefully peeled away and exhibited no oiliness or inhomogeneity unless otherwise noted. The film was cut into small pieces to be used for preparation of test bars by compression molding (size of pieces was similar to the hole dimensions of the mold plate). The film pieces were stacked into the holes of a multi-hole steel mold plate, pre-heated to 170° C., having hole dimensions 20 mm×12.8 mm×1.8 mm (ASTM D1693-95 dimensions). The mold plate was pressed in a PHI company QL-433-6-M2 model hydraulic press equipped with separate heating and cooling platforms. The upper and lower press plates were covered in Teflon™-coated aluminum foil and the following multistage press procedure was used at 170° C. with no release between stages: (1) three minutes with 1-2 ton overpressure; (2) one minute at 10 tons; (3) one minute at 15 tons; (4) three minutes at 30 tons; (5) release and three minutes in the cooling stage of the press (7° C.) at 30 tons. A knockout tool was then used to remove the sample bars with minimal flexion. Typically near-colorless, flexible bars were obtained which, when stored at room temperature, showed no oiliness or exudation several weeks after pressing unless otherwise noted.

### Illustrative Example 15

### Properties of PVC Bars Plasticized with Esters

Two each of the sample bars prepared in Example 14 were visually evaluated for appearance and clarity and further compared to identically prepared bars plasticized with DINP by placing the bars over a standard printed text. The qualitative and relative flexibility of the bars was also crudely evaluated by hand. The various bars were evaluated in different test batches; thus, a new DINP control bar was included with each batch. The bars were placed in aluminum pans which were then placed inside a glass crystallization dish covered with a watch glass. The bars were allowed to sit under ambient conditions at room temperature for at least three weeks and re-evaluated during and/or at the end of this aging period. Table 8 presents appearance rankings and notes.

TABLE 8
Initial and Room Temperature Aging Clarity and
Appearance Properties of Plasticized PVC Bars.

| Example No. | Initial | Final Clarity | |
|---|---|---|---|
| (Plasticizer | Clarity | Value (day of | Notes on Bar at |
| Used in Bar) | Value* | evaluation) | End of Test |
| 1 | — | — | — |
| 2 | — | — | — |
| 3 | 1^{b} | 1 (35) | Stiff |
| 4 | — | — | — |
| 5 | 1^{c} | 1 (29) | Slightly stiff |
| 6 | 1^{d} | —21 | Slightly stiff, ˜to DINP |
| 7 | 1^{d} | —(21) | Good flex (sl. > DINP) |
| 8 | 1^{d} | —(21) | Excellent flex (>DINP) |
| 9 | 1^{b} | 1 (35) | Moderately stiff |
| 10 | 1^{b} | 1 (35) | Stiff |
| C1 | 1^{b} | 1 (35) | Excellent flex (>DINP) |
| 12 | 1^{b} | 1 (35) | Excellent flex (>DINP) |
| DINP ctrl Ex. | 1^{b} | 1 (35) | Moderate flex |
| 3, 9, 10, C1, 12 | | | |
| DINP ctrl Ex. 5 | 1^{c} | 1 (29) | Light color |
| DINP ctrl Ex. | 1^{d} | —(21) | Somewhat stiff |
| 6, 7, 8 | | | |

— Data not taken.
*1-5 scale, 1 = no distortion, 5 = completely opaque; no bars exhibited oiliness, stickiness, or inhomogeneity unless noted.
Bars reflected color, if any, of neat plasticizers (3, 6, 7, 8, C1, 12 = light yellow; 9, 10 = light brown; 2 = light yellow and slightly cloudy).
^{b}Evaluation date not recorded.
^{c}Evaluated 3 days after pressing.
^{d}Evaluated 14 days after pressing.

### Illustrative Example 16

### 98° C. Weight Loss Study of Plasticized PVC Bars

Two each of the PVC sample bars prepared in Example 14 were placed separately in aluminum weighing pans and placed inside a convection oven at 98° C. Initial weight measurements of the hot bars and measurements taken at specified time intervals were recorded and results were averaged between the bars. The averaged results are shown in Table 9. Notes on the appearance and flexibility of the bars at the end of the test are also given. The final color of the bars (even DINP control samples) varied between batches; gross comparisons only should be made between bars of different test batches.

TABLE 9
% Weight Loss at 98° C. of Plasticized PVC Bars.

| Example | | | | | | | |
|---|---|---|---|---|---|---|---|
| No. | | | | | | | |
| (Plasti- | | | | | | | |
| cizer Used | Day | Day | Day | Day | Day | Day | |
| in Bar) | 1 | 2 | 3 | 7 | 14 | 21 | Notes on Bar^{a} |
| 1 | — | — | — | — | — | — | — |
| 2 | — | — | — | — | — | — | — |
| 3 | 0.24 | 0.32 | 0.37 | 0.34 | 0.38 | 0.42 | Near clear, stiff, |
| | | | | | | | almost brittle |
| 4 | — | — | — | — | — | — | — |
| 5 | 0.24 | 0.31 | 0.40 | 0.46 | 0.67 | 0.75 | Med brown, |
| | | | | | | | curled, still |
| | | | | | | | flexible |
| 6 | 0.35 | 0.42 | 0.70 | 0.77 | 1.22 | 1.62 | Med light brown, |
| | | | | | | | somewhat stiff |
| 7 | 0.30 | 0.38 | 0.68 | 0.65 | 1.10 | 1.48 | Very light brown, |
| | | | | | | | flex˜DINP |
| 8 | 0.46 | 0.64 | 1.31 | 1.48 | 2.64 | 3.47 | Light brown, |
| | | | | | | | flex˜DINP |
| 9 | 0.47 | 0.58 | 0.63 | 0.64 | 0.80 | 0.87 | Near clear, stiff, |
| | | | | | | | almost brittle |
| 10 | 0.58 | 0.75 | 0.81 | 0.80 | 0.84 | 0.88 | Near clear, stiff |
| | | | | | | | almost brittle |
| C1 | 0.27 | 0.33 | 0.42 | 0.47 | 0.63 | 0.72 | Yellow, excellent |
| | | | | | | | flex (>DINP) |
| 12 | 0.22 | 0.29 | 0.41 | 0.44 | 0.56 | 0.76 | Orange, good flex |
| | | | | | | | (sl. < DINP) |
| DINP ctrl | 0.20 | 0.27 | 0.31 | 0.36 | 0.48 | 0.56 | Dark, med brown, |
| Ex. 3, 9, | | | | | | | good flex |
| 10, C1, 12 | | | | | | | |
| DINP ctrl | 0.26 | 0.33 | 0.40 | 0.55 | 0.73 | 0.83 | Med brown, |
| Ex.5 | | | | | | | slight loss of flex |
| DINP ctrl | 0.31 | 0.42 | 0.43 | 0.48 | 0.64 | 0.74 | Light brown, |
| Ex. 6, 7, 8 | | | | | | | good flex |

Bars did not exhibit oiliness, stickiness, or inhomogeneity unless noted.
^{a}See notes in Table 8 regarding initial color of neat plasticizers and bars.

### Illustrative Example 17

### 70° C. Humid Aging Study of Plasticized PVC Bars

Using a standard one-hole office paper hole punch, holes were punched in two each of the sample bars prepared in Example 14 about ⅛″ from one end of the bar. The bars were hung in a glass pint jar (two bars per jar) fitted with a copper insert providing a stand and hook. The jar was filled with ˜½″ of distilled water and the copper insert was adjusted so that the bottom of each bar was ˜1″ above the water level. The jar was sealed, placed in a 70° C. convection oven, and further sealed by winding Teflon™ tape around the edge of the lid. After 21 days the jars were removed from the oven, allowed to cool for ˜20 minutes, opened, and the removed bars were allowed to sit under ambient conditions in aluminum pans (with the bars propped at an angle to allow air flow on both faces) or hanging from the copper inserts for ca. one week (until reversible humidity-induced opacity had disappeared). The bars were evaluated visually for clarity. All bars exhibited complete opacity during the duration of the test and for several days after removal from the oven. Results are shown in Table 10. Notes on the appearance and flexibility of the bars at the end of the test are also given.

TABLE 10
70° C. Humid Aging Clarity and Appearance
Properties of Plasticized PVC Bars.

| | Clarity Value | |
|---|---|---|
| | After Test* | |
| Example No. | (days aged at | |
| (Plasticizer Used in Bar) | ambient) | Notes on Bar |
| 1 | — | — |
| 2 | — | — |
| 3 | 1.5 (14) | Stiff |
| 4 | — | — |
| 5 | 1.5 (10) | Not brittle |
| 6 | 1 (14) | Slightly stiff, ˜DINP |
| 7 | 1 (14) | Slightly stiff, ˜DINP |
| 8 | 1 (14) | Extremely flexible, >>DINP |
| 9 | 1 (14) | Stiff |
| 10 | 2 (14) | Very stiff^{a} |
| C1 | 1 (14) | Excellent flex, slightly sticky |
| 12 | 1.5 (14) | Excellent flex (sl. < C1) |
| DINP ctrl Ex. 3, 9, | 1 (14) | Moderate flex |
| 10, C1, 12 | | |
| DINP ctrl Ex. 5 | 1.5 (10) | Very flexible |
| DINP ctrl Ex. 6, 7, 8 | 1 (14) | Slightly stiff |

*1-5 scale, 1 = no distortion, 5 = completely opaque. Bars did not exhibit oiliness, stickiness, or inhomogeneity unless noted.
^{a}This bar still showed some white spots from humidity-induced opacity after 14 days ambient aging.

### Illustrative Example 18

### Thermogravimetric Analysis (TGA) Property Study of Plasticized PVC Bars

The sample bars prepared in Example 14 were subjected to Thermogravimetric Analysis as described in Example 13 to evaluate plasticizer volatility in the formulated test bars. Table 11 provides a volatility comparison.

TABLE 11
Volatility Properties of Plasticizers in Plasticized PVC Bars.

| | | | | |
|---|---|---|---|---|
| Ex. No. | TGA 1% Wt | TGA 5% Wt | TGA 10% Wt | TGA Wt Loss |
| in Bar | Loss (° C.) | Loss (° C.) | Loss (° C.) | at 220° C. (%) |

| | | | | |
|---|---|---|---|---|
| Neat PVC | 129.9 | 192.3 | 255.4 | 6.3 |
| DINP | 204.6 | 247.4 | 257.6 | 1.8 |
| 1 | 224.4 | 248.5 | 259.5 | 0.8 |
| 2 | 210.5 | 243.3 | 254.4 | 1.4 |
| 3 | — | — | — | — |
| 4 | 205.7 | 243.5 | 256.4 | 1.7 |
| 5 | — | — | — | — |
| 6 | — | — | — | — |
| 7 | — | — | — | — |
| 8 | — | — | — | — |
| 9 | — | — | — | — |
| 10 | — | — | — | — |
| C1 | — | — | — | — |
| 12 | — | — | — | — |

— Data not taken.

### Illustrative Example 19

### Demonstration of Plasticization of PVC Via Differential Scanning calorimetry (DSC)

Differential Scanning Calorimetry (DSC) was performed on the compression-molded sample bars prepared in Example 13 using a TA Instruments 2920 calorimeter fitted with a liquid N₂ cooling accessory. Samples were loaded at room temperature and cooled to −90° C. at 10° C./min, and then analyzed on heating at a rate of 10° C./min to 150-170° C. for plasticized PVC bars, and to 100° C. for the comparative neat PVC bar. Small portions of the sample bars (typical sample mass 5-7 mg) were cut for analysis, making only vertical cuts perpendicular to the largest surface of the bar to preserve the upper and lower compression molding “skins”; the pieces were then placed in the DSC pans so that the upper and lower “skin” surfaces contacted the bottom and top of the pan. Table 12 provides the first heat T_{g} onset, midpoint, and end for neat PVC and the plasticized bars. A lowering and broadening of the glass transition for neat PVC is observed upon addition of the experimental plasticizers, indicating plasticization and extension of the flexible temperature range of use for neat PVC (for aid in calculating the numerical values of these broad transitions, the DSC curve for each plasticized bar was overlaid with the analogous Dynamic Mechanical Thermal Analysis (DMTA) curve, taken and analyzed as described in Example 20 (below), since the DMTA curve provides additional guidance about the proper temperature regions for the onset, midpoint, and end of T_{g}).

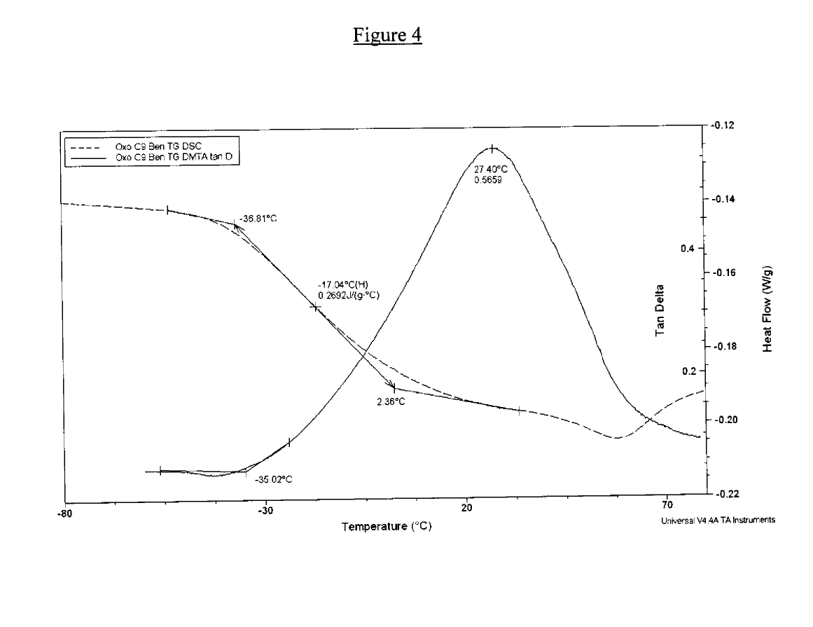

FIG. 4 is an overlay plot of the DMTA tan delta curve and the DSC curve for PVC plasticized with the triglyceride ester prepared in Example 1, showing correlation between the DSC and DMTA glass transition onsets.

TABLE 12
Glass Transition Onset, Midpoint, and End for Plasticized PVC Bars

| Ex. No. | T_{g} Onset | T_{g} Midpt | T_{g End} | Tₘ Max (° C.) |
|---|---|---|---|---|
| Used in Bar | (° C.) | (° C.) | (° C.) | and ΔH_{f} (J/g)^{a} |
| Neat PVC | 44.5 | 46.4 | 48.9 | not calc. |
| DINP | −37.8 | −24.8 | −12.2 | not calc. |
| 1 | −36.8 | −17.0 | 2.4 | not calc. |
| 2 | — | — | — | — |
| 3 | — | — | — | — |
| 4 | −27.5 | −8.5 | 10.5 | 58.7 (1.6) |
| 5 | — | — | — | — |
| 6 | — | — | — | — |
| 7 | — | — | — | — |
| 8 | — | — | — | — |
| 9 | — | — | — | — |
| 10 | — | — | — | — |
| Cl | — | — | — | — |
| 12 | — | — | — | — |

— Data not obtained.
^{a}Some sample bars showed a weak melting point (Tₘ) from the crystalline portion of PVC. Often this weak transition was not specifically analyzed, but data is given here in instances where it was recorded.

### Illustrative Example 20

### Demonstration of Plasticization of PVC Via Dynamic Mechanical Thermal Analysis (DMTA)

Three-point bend Dynamic Mechanical Thermal Analysis (DMTA) with a TA Instruments DMA Q980 fitted with a liquid N₂ cooling accessory and a three-point bend clamp assembly was used to measure the thermo-mechanical performance of neat PVC and the PVC/plasticizer blend sample bars prepared in Example 14. Samples were loaded at room temperature and cooled to −60° C. at a cooling rate of 3° C./min. After equilibration, a dynamic experiment was performed at one frequency using the following conditions: 3° C./min heating rate, 1 Hz frequency, 20 micrometer amplitude, 0.01 pre-load force, force track 120%. Two or three bars of each sample were typically analyzed; in the absence of other factors indicating data quality, numerical data was taken from the bar showing the lowest T_{g} onset. Glass transition onset values were obtained by extrapolation of the tan delta curve from the first deviation from linearity. The DMTA measurement gives storage modulus (elastic response modulus) and loss modulus (viscous response modulus); the ratio of loss to storage moduli at a given temperature is tan delta. The beginning (onset) of the T_{g} (temperature of brittle-ductile transition) was obtained for each sample by extrapolating a tangent from the steep inflection of the tan delta curve and the first deviation of linearity from the baseline prior to the beginning of the peak. Table 13 provides a number of DMTA parameters for neat PVC and PVC bars plasticized with the esters of the invention: T_{g} onset (taken from tan delta); peak of the tan delta curve; storage modulus at 25° C.; and the temperature at which the storage modulus equals 100 MPa (this temperature was chosen to provide an arbitrary measure of the temperature at which the PVC loses a set amount of rigidity; too much loss of rigidity may lead to processing complications for the PVC material). The flexible use temperature range of the plasticized PVC samples is evaluated as the range between the T_{g} onset and the temperature at which the storage modulus was 100 MPa. A lowering and broadening of the glass transition for neat PVC is observed upon addition of the experimental plasticizers, indicating plasticization and extension of the flexible temperature range of use for neat PVC. Plasticization (enhanced flexibility) is also demonstrated by lowering of the PVC room temperature storage modulus.

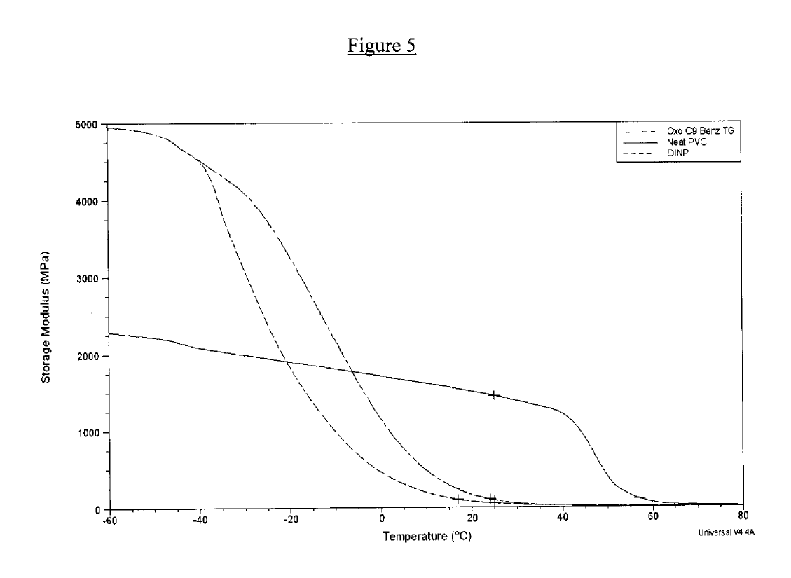

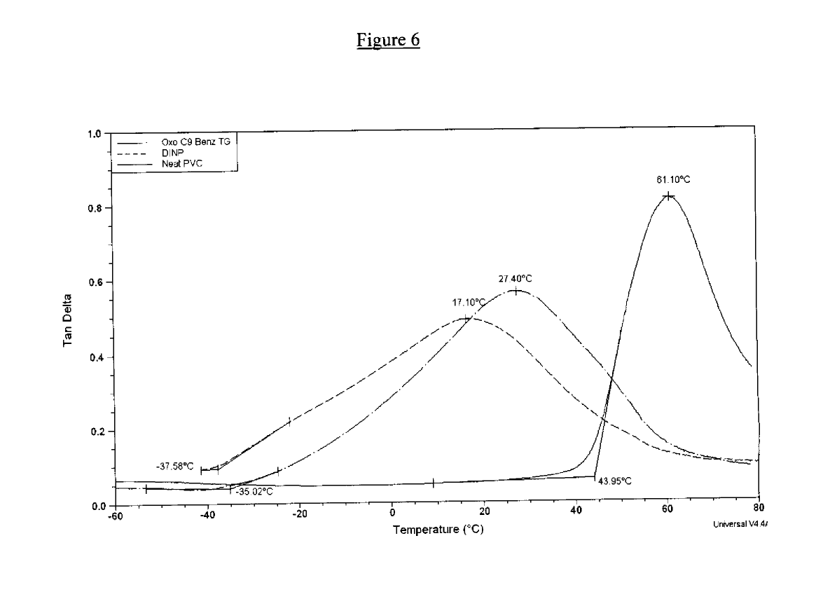

FIG. 5 is an overlay plot of the Dynamic Mechanical Thermal Analysis (DMTA) storage modulus curves for (a) neat PVC, (b) PVC plasticized with the commercial phthalate DINP, and (c) PVC plasticized with the triglyceride ester prepared in Example 1 (“Oxo C9 Benz TG”). The cross points marked on the curves indicate the points at which the numerical data given in Table 13 was obtained (temperature of 100 MPa storage modulus and storage modulus at 25° C.). FIG. 6 is an overlay plot of DMTA tan delta curves for (a) neat PVC, (b) PVC plasticized with the commercial phthalate DINP, and (c) PVC plasticized with the triglyceride ester prepared in Example 1 (“Oxo C9 Benz TG”). The glass transition onset temperature and temperature of peak tan delta curve (given in Table 13) are labeled for each curve.

TABLE 13
Various DMTA Thermal Parameters for Plasticized PVC Bars

| | | | | Temp. | |
|---|---|---|---|---|---|
| Ex. | Tan Δ T_{g} | Tan Δ | 25° C. | of 100 | Flexible |
| No. Used | Onset | Peak | Storage | MPa Storage | Use Range |
| in Bar | (° C.) | (° C.) | Mod. (MPa) | Mod. (° C.) | (° C.)^{a} |
| Neat PVC | 44.0 | 61.1 | 1433 | 57.1 | 13.1 |
| DINP | −37.6 | 17.1 | 48.6 | 16.9 | 54.5 |
| 1 | −35.0 | 27.4 | 89.8 | 24.0 | 59.0 |
| 2 | −35.9 | 24.4 | 95.0 | 24.7 | 60.6 |
| 3 | — | — | — | — | — |
| 4 | −23.3 | 23.3 | 82.4 | 24.1 | 47.4 |
| 5 | — | — | — | — | — |
| 6 | — | — | — | — | — |
| 7 | — | — | — | — | — |
| 9 | — | — | — | — | — |
| 10 | — | — | — | — | — |
| C1 | — | — | — | — | — |
| 12 | — | — | — | — | — |

— Data not obtained.
^{aDifference between temperature of 100 MPa storage modulus and onset of T}_{g}.

### Illustrative Example 21

### Further Demonstration of PVC Plasticization with Mixed Triglycerides

Plasticized PVC samples containing the triglycerides of Examples 1, 4, or 6 or DINP (as a comparative) were mixed at room temperature with moderate stirring, then placed on a roll mill at 340° F. and milled for 6 minutes. The flexible vinyl sheet was removed and compression molded at 340° F. The samples had the following formulation: 100 phr Oxy Vinyls® 240 PVC resin; 50 phr triglyceride or DINP; 2.5 phr epoxidized soybean oil; 2.5 phr Mark® 1221 Ca/Zn stabilizer; 0.3 phr stearic acid. Comparison of the data for the formulations is given in Table 14.

TABLE 14
Properties of PVC Samples Plasticized With 50 phr
Mixed Triglycerides Versus DINP

| | Ex. 6 | Ex. 4 | Ex. 1 | |
|---|---|---|---|---|
| Plasticizer Used in Formulation | C₆/Bz | C₇/Bz | C₉/Bz | DINP^{a} |
| Original Mechanical Properties | | | | |
| Shore A Hardness (15 sec.) | 77.3 | 80.5 | 83.6 | 80.3 |
| 95% Confidence Interval | 0.4 | 0.8 | 0.5 | — |
| Shore D Hardness (15 sec.) | 27.3 | 30.2 | 33.1 | — |
| 95% Confidence Interval | 0.2 | 0.5 | 0.2 | — |
| 100% Modulus Strength, psi | 1941 | 2111 | 2295 | 1691 |
| 95% Confidence Interval | 59 | 27 | 27 | — |
| Ultimate Tensile Strength, psi | 3422 | 3537 | 3349 | 3267 |
| 95% Confidence Interval | 133 | 81 | 61 | — |
| Ultimate Elongation, % | 318 | 318 | 315 | 367 |
| 95% Confidence Interval | 14 | 5 | 20 | — |
| Aged Mechanical Properties | 70° C. | 100° C. | 100° C. | 100° C. |
| (7 days at given temp., AC/hour) | | | | |
| Aged 100% Modulus Strength, psi | 1984 | 2724 | 2378 | 2390 |
| 95% Confidence Interval | 32 | 44 | 27 | — |
| Ultimate Tensile Strength, psi | 3395 | 3391 | 3402 | 3013 |
| 95% Confidence Interval | 84 | 68 | 81 | — |
| Ultimate Elongation, % | 317 | 296 | 321 | 267 |
| 95% Confidence Interval | 12 | 11 | 15 | — |
| Weight Loss, Wt % | 1.2 | 0.2 | 1.7 | 7.0 |
| 95% Confidence Interval | 0.04 | 0.11 | 0.09 | — |
| Retained Properties | 70° C. | 100° C. | 100° C. | 100° C. |
| (7 days at given temp., AC/hour) | | | | |
| Retained 100% Modulus | 102 | 129 | 104 | 141 |
| Strength, % | | | | |
| 95% Confidence Interval | 0.4 | 0.4 | 0.3 | — |
| Retained Tensile Strength, % | 99 | 96 | 102 | 92 |
| 95% Confidence Interval | 0.4 | 0.3 | 0.3 | — |
| Retained Elongation, % | 100 | 93 | 102 | 73 |
| 95% Confidence Interval | 1.3 | 1 | 1.6 | 13 |
| Low Temperature | | | | |
| Clash Berg (T_{f}), ° C. | −7.4 | −5.8 | −6.3 | −21.0 |
| 95% Confidence Interval | 1.6 | 0.9 | 1.9 | — |

— = Data unavailable.
^{a}Similar formulation tested separately: 50 phr DINP, 3.0 phr Epoxidized Soybean Oil, 2.5 phr Mark 1221, 0.25 phr stearic acid.

All patents and patent applications, test procedures (such as ASTM methods, UL methods, and the like), and other documents cited herein are fully incorporated by reference to the extent such disclosure is not inconsistent with this invention and for all jurisdictions in which such incorporation is permitted.

When numerical lower limits and numerical upper limits are listed herein, ranges from any lower limit to any upper limit are contemplated. While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth herein but rather that the claims be construed as encompassing all the features of patentable novelty which reside in the present invention, including all features which would be treated as equivalents thereof by those skilled in the art to which the invention pertains.

The invention has been described above with reference to numerous embodiments and specific examples. Many variations will suggest themselves to those skilled in this art in light of the above detailed description. All such obvious variations are within the full intended scope of the appended claims.

What is claimed is:

1. A process for producing a plasticizer comprising:
(i) recovering at least one linear C₄ to C₁₃ aldehyde, one branched C₄ to C₁₃ aldehyde, or a combination thereof from a hydroformylation product;
(ii) oxidizing the linear, branched or combination thereof C₄ to C₁₃ aldehyde to form a linear, branched or combination thereof C₄ to C₁₃ acid;
(iii) combining the linear, branched or combination thereof C₄ to C₁₃ acid with benzoic acid, toluic acid or a combination thereof at a molar ratio ranging from 0.25:1 to 4:1 to form a mixed acid blend;
(iv) esterifying the mixed acid blend with a glycerol to yield a linear alkyl-aryl triglyceride, a branched alkyl-aryl triglyceride, or a combination thereof, and
(v) purifying the linear, branched or combination thereof alkyl-aryl triglyceride to form a plasticizer, wherein the total carbon number of the triester groups ranges from 20 to 25 and includes from 1 to 2 aryl groups for greater than or equal to 45 wt % of the plasticizer.

2. The process of claim 1, wherein the molar ratio of C₄ to C₁₃ acid to benzoate and/or toluate in the mixed acid blend ranges from 0.33:1 to 3:1.

3. The process of claim 1, wherein the molar ratio of C₄ to C₁₃ acid to benzoate and/or toluate in the mixed acid blend ranges from 0.5:1 to 2:1.

4. The process of claim 1, wherein the molar ratio of C₄ to C₁₃ acid to benzoate and/or toluate in the mixed acid blend ranges from 0.67:1 to 1.5:1.

5. The process of claim 1, wherein the molar ratio of C₄ to C₁₃ acid to benzoate and/or toluate in the mixed acid blend is 1:1.

6. The process of claim 1, wherein the toluic acid includes the ortho isomer, the meta isomer, the para isomer, and combinations thereof.

7. The process of claim 1, wherein the at least one branched C₄ to C₁₃ aldehyde is characterized by a branching of from about 0.2 to about 4.0 branches per molecule.

8. The process of claim 1, wherein the oxidizing step is with oxygen and/or air.

9. The process of claim 1, further including recovering at least one linear C₄ to C₁₃ alcohol, one branched C₄ to C₁₃ alcohol, or a combination thereof from the hydroformylation product, oxidizing the linear, branched or combination thereof C₄ to C₁₃ alcohol to form a linear, branched or combination thereof C₄ to C₁₃ acid; and feeding the linear, branched or combination thereof C₄ to C₁₃ acid to steps (iii) through (v) of claim 1.

10. The process of claim 1, wherein the hydroformylation product includes a mixture of at least one linear C₄ to C₁₃ aldehyde, one branched C₄ to C₁₃ aldehyde, or a combination thereof and at least one at least one linear C₄ to C₁₃ alcohol, one branched C₄ to C₁₃ alcohol, or a combination thereof.

11. The process of claim 1, further including purifying the linear, branched or combination thereof C₄ to C₁₃ acid of step (ii) from the unreacted linear, branched or combination thereof C₄ to C₁₃ aldehyde via distillation before the combining step (iii).

12. The process of claim 1, wherein the glycerol is crude glycerol chosen from REG, EIS-739, EIS-740, EIS-733, EIS-724, EIS 56-81-5, IRE and mixtures thereof.

13. The process of claim 12, wherein the crude glycerol includes from 50 wt % to 95 wt % glycerol.

14. The process of claim 1, further comprising providing a feed for the hydroformylation reaction from dimerization of a feedstock.

15. The process of claim 14, wherein the feedstock comprises an olefin selected from propylene, butenes, pentenes and mixtures thereof.

16. The process of claim 14, wherein the hydroformylation reaction is catalyzed by a metal selected from Groups 8-10 according to the new notation for the Periodic Table as set forth in Chemical Engineering News, 63(5), 27 (1985).

17. The process of claim 16, wherein the hydroformylation reaction is catalyzed by a metal selected from Rh, Co, and mixtures thereof.

18. The process of claim 17, wherein the hydroformylation reaction is catalyzed by a metal selected from Rh, Co, and mixtures thereof including an organic ligand.

19. The process of claim 1, wherein the branched C₄ to C₁₃ acid is an Oxo acid or a Neo acid.

20. The process of claim 1, wherein the total carbon number of the ester groups ranges from 20 to 25 and includes from 1 to 2 aryl groups for greater than or equal to 70 wt % of the plasticizer.

21. The process of claim 1, wherein the total carbon number of the ester groups ranges from 20 to 25 and includes from 1 to 2 aryl groups for greater than or equal to 95 wt % of the plasticizer.

22. The process of claim 1, wherein the oxidizing step is catalyzed.

23. The process of claim 1, wherein the oxidizing step is not catalyzed.

24. The process of claim 1, wherein the esterifying step is catalyzed by at least one metal selected from Ti, Zr or Sn, or a mixture thereof, or catalyzed by an organic acid.

25. The process of claim 1, further comprising dimerizing a feedstock selected from propylene, butenes, pentenes and mixtures thereof by solid phosphoric acid or a zeolite dimerization to provide a feed for the hydroformylation reaction.

26. A plasticizer made by the process of claim 1.

27. The plasticizer of claim 26 characterized as being phthalate-free.

28. A resin composition comprising the plasticizer of claim 26 and a resin.

29. The resin composition of claim 28, wherein the resin is selected from polyvinyl chloride, polyesters, polyurethanes, ethylene-vinyl acetate copolymer, rubbers, acrylics, and mixtures thereof.

30. The resin composition of claim 28, further comprising stabilizers, fillers, pigments, biocides, carbon black, adhesion promoters, viscosity reducers, thixotropic agents, thickening agents, blowing agents, and mixtures thereof.

31. The resin composition of claim 28, further comprising at least one plasticizer selected from phthalates, adipates, trimellitates, cyclohexanoates, benzoates, and combinations thereof.

32. A plastisol comprising the plasticizer of claim 26.

33. An article comprising the plasticizer of claim 26, the resin composition of claim 28, or the plastisol of claim 32.

34. The article of claim 33, wherein the article is selected from toys, films and sheets, tubing, coated fabrics, wire and cable insulation and jacketing, flooring materials, preferably vinyl sheet flooring or vinyl floor tiles, adhesives, sealants, inks, and medical products, preferably blood bags and medical tubing.

35. The article of claim 34, made by a process including steps of dryblending and extrusion.

36. A plasticizer comprising a triglyceride according to the formula 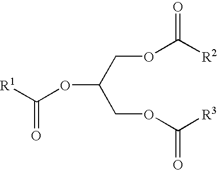
wherein each of R¹, R², and R³ are independently selected from a combination of C₃ to C₁₂ linear or branched alkyl groups and aryl groups, and wherein the total carbon number of the triester groups ranges from 20 to 25, and wherein the aryl groups are selected from benzoate groups, toluate groups and combinations thereof, and wherein the molar ratio of C₃ to C₁₂ linear or branched alkyl groups to benzoate and/or toluate groups in the triglyceride ranges from 0.5:1 to 2:1.

37. The plasticizer of claim 36, wherein the molar ratio of C₃ to C₁₂ linear or branched alkyl groups to benzoate and/or toluate groups in the triglyceride ranges from 1:1 to 2:1.

38. The plasticizer of claim 36, wherein the molar ratio of C₃ to C₁₂ linear or branched alkyl groups to benzoate and/or toluate groups in the triglyceride ranges from 1.4:1 to 2:1.

39. The plasticizer of claim 36, wherein the molar ratio of C₃ to C₁₂ linear or branched alkyl groups to benzoate and/or toluate groups in the triglyceride ranges from 0.5:1 to 1.4:1.

40. The plasticizer of claim 36, wherein the molar ratio of C₃ to C₁₂ linear or branched alkyl groups to benzoate and/or toluate groups in the triglyceride ranges from 0.5:1 to 1:1.

41. The plasticizer of claim 36, wherein one or two of the R¹, R², and R³ groups is a linear or branched C₅ alkyl group, and one or two of the R¹, R², and R³ groups is a benzoate group, toluate group or combinations thereof.

42. The plasticizer of claim 36, wherein one or two of the R¹, R², and R³ groups is a linear or branched C₆ alkyl group, and one or two of the R¹, R², and R³ groups is a benzoate group, toluate group or combinations thereof.

43. The plasticizer of claim 36, wherein one or two of the R¹, R², and R³ groups is a linear or branched C₇ alkyl group, and one or two of the R¹, R², and R³ groups is a benzoate group, toluate group or combinations thereof.

44. The plasticizer of claim 36, wherein one or two of the R¹, R², and R³ groups is a linear or branched C₈ alkyl group, and one or two of the R¹, R², and R³ groups is a benzoate group, toluate group or combinations thereof.

45. The plasticizer of claim 36, wherein one or two of the R¹, R², and R³ groups is a linear or branched C₉ alkyl group, and one or two of the R¹, R², and R³ groups is a benzoate group, toluate group or combinations thereof.

46. The plasticizer of claim 36, wherein R¹, R², and R³ comprise one C₅ linear or branched alkyl group, one C₈ linear or branched alkyl group, and one benzoate or toluate group.

47. The plasticizer of claim 36, wherein R¹, R², and R³ comprise one C₄ linear or branched alkyl group, one C₉ linear or branched alkyl group, and one benzoate or toluate group.

48. The plasticizer of claim 36, wherein R¹, R², and R³ comprise one C₅ linear or branched alkyl group, one C₆ linear or branched alkyl group, and one benzoate or toluate group.

49. The plasticizer of claim 36, wherein the average branching of the C₃ to C₁₂ branched alkyl groups is from about 0.2 to about 4.0 branches per group.

50. The plasticizer of claim 36, wherein the triglyceride comprises greater than or equal to 45 wt % of the plasticizer.

51. The plasticizer of claim 48, wherein the triglyceride comprises greater than or equal to 70 wt % of the plasticizer.

52. The plasticizer of claim 49, wherein the triglyceride comprises greater than or equal to 95 wt % of the plasticizer.

53. The plasticizer of claim 50, wherein one or two of R¹, R², and R³ are aryl groups in at least 45 wt % of the triglycerides.

54. The plasticizer of claim 51, wherein one or two of R¹, R², and R³ are aryl groups in at least 70 wt % of the triglycerides.

55. The plasticizer of claim 52, wherein one or two of R¹, R², and R³ are aryl groups in at least 95 wt % of the triglycerides.

56. A plasticizer comprising a blend of two or more different triglycerides according to claim 36.

57. The plasticizer of claim 56, wherein the blend comprises greater than or equal to 45 wt % of the plasticizer.

58. The plasticizer of claim 57, wherein the blend comprises greater than or equal to 70 wt % of the plasticizer.

59. The plasticizer of claim 58, wherein the blend comprises greater than or equal to 95 wt % of the plasticizer.

60. The plasticizer of claim 57, wherein one or two of R¹, R², and R³ are aryl groups in at least 45 wt % of the triglycerides in the blend.

61. The plasticizer of claim 58, wherein one or two of R¹, R², and R³ are aryl groups in at least 70 wt % of the triglycerides in the blend.

62. The plasticizer of claim 59, wherein one or two of R¹, R², and R³ are aryl groups in at least 95 wt % of the triglycerides in the blend.

63. The plasticizer of claim 36 characterized as being phthalate-free.

64. A resin composition comprising the plasticizer of claim 36 or claim 56 and a resin.

65. The resin composition of claim 64, wherein the resin is selected from polyvinyl chloride, polyesters, polyurethanes, ethylene-vinyl acetate copolymer, rubbers, acrylics, and mixtures thereof.

66. A plastisol comprising the plasticizer of claim 36 or claim 56.

67. An article comprising the plasticizer of claim 36 or claim 56, the resin composition of claim 64, or the plastisol of claim 66.

68. The article of claim 67, wherein the article is selected from toys, films and sheets, tubing, coated fabrics, wire and cable insulation and jacketing, flooring materials, preferably vinyl sheet flooring or vinyl floor tiles, adhesives, sealants, inks, and medical products, preferably blood bags and medical tubing.

69. A process for producing a plasticizer comprising:
(i) recovering an aldehyde/alcohol mixture including at least one linear C₄ to C₁₃ aldehyde, one branched C₄ to C₁₃ aldehyde, or a combination thereof and at least one linear C₄ to C₁₃ alcohol, one branched C₄ to C₁₃ alcohol, or a combination thereof from a hydroformylation process;
(ii) oxidizing the aldehyde/alcohol mixture to form a linear, branched or combination thereof C₄ to C₁₃ acid;
(iii) combining the linear, branched or combination thereof C₄ to C₁₃ acid with benzoic acid, toluic acid or a combination thereof at a molar ratio ranging from 0.25:1 to 4:1 to form a mixed acid blend;
(iv) esterifying the mixed acid blend with glycerol to yield a linear alkyl-aryl triglyceride, a branched alkyl-aryl triglyceride, or a combination thereof; and
(v) purifying the linear, branched or combination thereof alkyl-aryl triglyceride to form a plasticizer, wherein the total carbon number of the triester groups ranges from 20 to 25 and includes from 1 to 2 aryl groups for greater than or equal to 45 wt % of the plasticizer.

70. The process of claim 69, wherein the molar ratio of C₄ to C₁₃ acid to benzoate and/or toluate in the mixed acid blend ranges from 0.33:1 to 3:1.

71. The process of claim 69, wherein the molar ratio of C₄ to C₁₃ acid to benzoate and/or toluate in the mixed acid blend ranges from 0.5:1 to 2:1.

72. The process of claim 69, wherein the molar ratio of C₄ to C₁₃ acid to benzoate and/or toluate in the mixed acid blend ranges from 0.67:1 to 1.5:1.

73. The process of claim 69, wherein the molar ratio of C₄ to C₁₃ acid to benzoate and/or toluate in the mixed acid blend is 1:1.

74. The process of claim 69, wherein the toluic acid includes the ortho isomer, the meta isomer, the para isomer, and combinations thereof.

75. The process of claim 69, further including purifying the aldehyde/alcohol mixture of step (i) via distillation before the oxidizing step (ii).

76. The process of claim 69, wherein the at least one branched C₄ to C₁₃ aldehyde is characterized by a branching of from about 0.2 to about 4.0 branches per molecule.

77. The process of claim 69, wherein the oxidizing step is with oxygen and/or air.

78. The process of claim 69, further including purifying the linear, branched or combination thereof C₄ to C₁₂ acid of step (ii) from the unreacted aldehyde/alcohol mixture via distillation before the combining step (iii).

79. The process of claim 69, wherein the glycerol is crude glycerol chosen from REG, EIS-739, EIS-740, EIS-733, EIS-724, EIS 56-81-5, IRE and mixtures thereof.

80. The process of claim 79, wherein the crude glycerol includes from 50 wt % to 95 wt % glycerol.

81. The process of claim 69, further comprising providing a feed for the hydroformylation process from dimerization of a feedstock.

82. The process of claim 81, wherein the feedstock comprises an olefin selected from propylene, butenes, pentenes and mixtures thereof.

83. The process of claim 82, wherein the hydroformylation process is catalyzed by a metal selected from Groups 8-10 according to the new notation for the Periodic Table as set forth in Chemical Engineering News, 63(5), 27 (1985).

84. The process of claim 83, wherein the hydroformylation process is catalyzed by a metal selected from Rh, Co, and mixtures thereof.

85. The process of claim 84, wherein the hydroformylation process is catalyzed by a metal selected from Rh, Co, and mixtures thereof including an organic ligand.

86. The process of claim 69, wherein the branched C₄ to C₁₃ acid is an Oxo acid or a Neo acid.

87. The process of claim 69, wherein the total carbon number of the ester groups ranges from 20 to 25 and includes from 1 to 2 aryl groups for greater than or equal to 70 wt % of the plasticizer.

88. The process of claim 69, wherein the total carbon number of the ester groups ranges from 20 to 25 and includes from 1 to 2 aryl groups for greater than or equal to 95 wt % of the plasticizer.

89. The process of claim 69, wherein the oxidizing step is catalyzed.

90. The process of claim 69, wherein the oxidizing step is not catalyzed.

91. The process of claim 69, wherein the esterifying step is catalyzed by at least one metal selected from Ti, Zr or Sn, or a mixture thereof, or catalyzed by an organic acid.

92. The process of claim 69, further comprising dimerizing a feedstock selected from propylene, butenes, pentenes and mixtures thereof by solid phosphoric acid or a zeolite dimerization to provide a feed for the hydroformylation process.

93. A plasticizer made by the process of claim 69.

94. The plasticizer of claim 93 characterized as being phthalate-free.

95. A resin composition comprising the plasticizer of claim 93 and a resin.

96. The resin composition of claim 95, wherein the resin is selected from polyvinyl chloride, polyesters, polyurethanes, ethylene-vinyl acetate copolymer, rubbers, acrylics, and mixtures thereof.

97. The resin composition of claim 95, further comprising stabilizers, fillers, pigments, biocides, carbon black, adhesion promoters, viscosity reducers, thixotropic agents, thickening agents, blowing agents, and mixtures thereof.

98. The resin composition of claim 95, further comprising at least one plasticizer selected from phthalates, adipates, trimellitates, cyclohexanoates, benzoates, and combinations thereof.

99. A plastisol comprising the plasticizer of claim 93.

100. An article comprising the plasticizer of claim 93, the resin composition of claim 95, or the plastisol of claim 99.

101. The article of claim 100, wherein the article is selected from toys, films and sheets, tubing, coated fabrics, wire and cable insulation and jacketing, flooring materials, preferably vinyl sheet flooring or vinyl floor tiles, adhesives, sealants, inks, and medical products, preferably blood bags and medical tubing.

102. The article of claim 101, made by a process including steps of dryblending and extrusion.

103. The resin composition of claim 64 wherein the hydrolysis products of the composition following melt processing as measured by gas chromatography are less than 0.5 wt % of the mixture.

104. The resin composition of claim 64 wherein the hydrolysis products of the composition following melt processing as measured by gas chromatography are less than 0.1 wt % of the mixture.

105. The resin composition of claim 95 wherein the hydrolysis products of the composition following melt processing as measured by gas chromatography are less than 0.5 wt % of the mixture.

106. The resin composition of claim 95 wherein the hydrolysis products of the composition following melt processing as measured by gas chromatography are less than 0.1 wt % of the mixture.
